(19) United States
(12) Patent Application Publication
(10) Pub. No.: US 2026/0335536 A1
(43) Pub. Date: Oct. 29, 2026

(54) Peripheral Device for Infusion Pump

(71) Applicant: Becton, Dickinson and Company, Franklin Lakes, NJ (US)

(72) Inventors: Ashley Rachel Rothenberg, Morris Plains, NJ (US); Zachary Traina, Denville, NJ (US); Mark Jesser, Wyckoff, NJ (US); Erik Kurt Witt, Oakland, NJ (US); Ami Kumordzie, New York, NY (US)

(21) Appl. No.: 19/117,800

(22) PCT Filed: Oct. 2, 2023
(86) PCT No.: PCT/US23/75677

§ 371 Date: Apr. 2, 2025

Related U.S. Application Data
(60) Provisional application No. 63/412,656, filed on Oct. 3, 2022.

(51) Int. Cl. A61M 5/142; A61M 5/168

(52) U.S. Cl. CPC A61M 5/142; A61M 5/16831; A61M 2205/3327; A61M 2205/3334; A61M 2205/3375; A61M 2205/3592; A61M 2205/502; A61M 2205/583; A61M 2205/6072; A61M 2205/8206

(57) ABSTRACT

A peripheral device for an infusion pump may include a housing and a mechanical interface configured to connect the housing to an exterior surface of the infusion pump. The housing may include a sensor configured to measure a parameter associated with the infusion pump and a wireless communication device configured to communicate, to an external device, information associated with the measured parameter of the infusion pump. A system may include the peripheral device and one or more processors programmed and/or configured to determine, based on the measured parameter of the infusion pump, a state of the infusion pump.

### CROSS-REFERENCE TO RELATED APPLICATOIN

The present application claims priority to U.S. Provisional Application Ser. No. 63/412,656, entitled “Peripheral Device for Infusion Pump” filed Oct. 3, 2022, the entire disclosure of which is hereby incorporated by reference in its entirety.

### BACKGROUND

Drug Infusion into a human body is often achieved through catheters that are either inserted into a peripheral vein (PIVC) or central vein (PICC/CVC). A catheter may be connected to an infusion pump via a needleless connector.

A patient may be connected to multiple lumens or fluid lines (e.g., IV lines, etc.). For example, a patient may be connected to multiple channels of an infusion pump system including a plurality of infusion pumps. As an example, a patient in an emergency room may be connected to ten or more fluid lines at the same time. The multiple fluid lines may result in confusion as to which fluid line at the patient connects to which infusion pump or fluid source. For example, when line maintenance is ordered, a nurse may have to trace a fluid line from a connection of the fluid line at an infusion pump or fluid source to a connection of the fluid line at the patient, or vice-versa. However, when multiple fluid lines are crisscrossed, overlapping, and/or tangled, it may be difficult to identify a correct line for maintenance, infusion, removal, and/or the like.

### SUMMARY

According to some non-limiting embodiments or aspects, provided is a peripheral device for an infusion pump, including: a housing including: a sensor configured to measure a parameter associated with the infusion pump; a wireless communication device configured to communicate, to an external device, information associated with the measured parameter of the infusion pump; and a mechanical interface configured to connect the housing to an exterior surface of the infusion pump.

According to some non-limiting embodiments or aspects, provided is a system including: a peripheral device for an infusion pump, including: a housing including: a sensor configured to measure a parameter associated with the infusion pump; a wireless communication device configured to communicate, to an external device, information associated with the measured parameter of the infusion pump; and a mechanical interface configured to connect the housing to an exterior surface of the infusion pump; and one or more processors programmed and/or configured to determine, based on the measured parameter of the infusion pump, a state of the infusion pump.

Further non-limiting embodiments or aspects are set forth in the following numbered clauses:

Clause 1. A peripheral device for an infusion pump, comprising: a housing including: a sensor configured to measure a parameter associated with the infusion pump; a wireless communication device configured to communicate, to an external device, information associated with the measured parameter of the infusion pump; and a mechanical interface configured to connect the housing to an exterior surface of the infusion pump.

Clause 2. The peripheral device of clause 1, wherein the housing is removably connected to the mechanical interface.

Clause 3. The peripheral device of any of clauses 1 and 2, wherein the housing is integrated with the mechanical interface.

Clause 4. The peripheral device of any of clauses 1-3, wherein the mechanical interface includes a base plate configured to be connected to the exterior surface of the infusion pump.

Clause 5. The peripheral device of any of clauses 1-4, wherein the mechanical interface includes an adhesive layer.

Clause 6. The peripheral device of any of clauses 1-5, wherein the mechanical interface includes a snap fit connector.

Clause 7. The peripheral device of any of clauses 1-6, wherein the housing further includes a battery configured to provide power to the sensor and the wireless communication device.

Clause 8. The peripheral device of any of clauses 1-7, wherein the battery includes a rechargeable battery.

Clause 9. The peripheral device of any of clause 1-8, wherein the housing includes a cover and a base, and wherein the cover is rotatable about an pin connecting the cover to the base.

Clause 10. The peripheral device of any of clauses 1-9, wherein the sensor includes at least one of the following: a button configured for mechanical interaction with a door of the infusion pump, a piezoelectric sensor, a tilt switch, an accelerometer, or any combination thereof.

Clause 11. The peripheral device of any of clauses 1-10, wherein the housing further includes one or more processors programmed and/or configured to determine, based on the measured parameter, a door open state and a door closed state of the door of the infusion pump.

Clause 12. The peripheral device of any of clauses 1-11, wherein the sensor includes an acoustic or vibration sensor configured to measure a noise or vibration of the infusion pump.

Clause 13. The peripheral device of any of clauses 1-12, wherein the housing further includes one or more processors programmed and/or configured to determine, based on the measured noise or vibration of the infusion pump, a pump on state and a pump off state of the infusion pump.

Clause 14. The peripheral device of any of clauses 1-13, wherein the wireless communication device is configured to receive, from a smart device connected to an IV line connected to the infusion pump, sensor data associated with at least one of a flow rate and a pressure of a fluid in the IV line, and wherein the one or more processors are programmed and/or configured to determine, based on the measured noise or vibration of the infusion pump and the at least one of the flow rate and the pressure of the fluid in the IV line, an unintended flow of the fluid in the IV line.

Clause 15. The peripheral device of any of clauses 1-14, wherein the housing further includes one or more processors programmed and/or configured to determine, based on the measured noise or vibration of the infusion pump, a migration or a dislodgement of an IV line connected to the infusion pump.

Clause 16. The peripheral device of any of clauses 1-15, wherein the housing further includes: a user input component configured to receive a user input from a user.

Clause 17. The peripheral device of any of clauses 1-16, wherein the housing further includes a user feedback component configured to provide feedback to the user.

Clause 18. The peripheral device of any of clauses 1-17, wherein the user feedback component is configured to produce a same type of visual output as a smart device connected to an IV line connected to the infusion pump based on a communication connection established between the wireless communication device and the smart device.

Clause 19. The peripheral device of any of clauses 1-18, wherein the housing further includes an identifier element encapsulating a unique identifier associated with the peripheral device, and wherein the identifier element includes at least one of the following: a barcode, an RFID tag, or any combination thereof.

Clause 20. A system comprising: the peripheral device of any of clauses 1-19; and the infusion pump, wherein the peripheral device is connected to the infusion pump.

Clause 21. A system comprising: a peripheral device for an infusion pump, including: a housing including: a sensor configured to measure a parameter associated with the infusion pump; a wireless communication device configured to communicate, to an external device, information associated with the measured parameter of the infusion pump; and a mechanical interface configured to connect the housing to an exterior surface of the infusion pump; and one or more processors programmed and/or configured to determine, based on the measured parameter of the infusion pump, a state of the infusion pump.

Clause 22. The system of clause 21, wherein the housing is removably connected to the mechanical interface.

Clause 23. The system of any of clauses 21 and 22, wherein the housing is integrated with the mechanical interface.

Clause 24. The system of any of clauses 21-23, wherein the mechanical interface includes a base plate configured to be connected to the exterior surface of the infusion pump.

Clause 25. The system of any of clauses 21-24, wherein the mechanical interface includes an adhesive layer.

Clause 26. The system of any of clauses 21-25, wherein the mechanical interface includes a snap fit connector.

Clause 27. The system of any of clauses 21-26, wherein the housing further includes a battery configured to provide power to the sensor, the wireless communication device, and the one or more processors.

Clause 28. The system of any of clauses 21-27, wherein the battery includes a rechargeable battery.

Clause 29. The system of any of clauses 21-28, wherein the housing includes a cover and a base, and wherein the cover is rotatable about an pin connecting the cover to the base.

Clause 30. The system of any of clauses 21-29, wherein the sensor includes at least one of the following: a button configured for mechanical interaction with a door of the infusion pump, a piezoelectric sensor, a tilt switch, an accelerometer, or any combination thereof.

Clause 31. The system of any of clauses 21-30, wherein the one or more processors are further programmed and/or configured to determine, based on the measured parameter, a door open state and a door closed state of the door of the infusion pump.

Clause 32. The system of any of clauses 21-31, wherein the sensor includes an acoustic or vibration sensor configured to measure a noise or vibration of the infusion pump.

Clause 33. The system of any of clauses 21-32, wherein the one or more processors are further programmed and/or configured to determine, based on the measured noise or vibration of the infusion pump, a pump on state and a pump off state of the infusion pump.

Clause 34. The system of any of clauses 21-33, further comprising: a smart device connected to an IV line connected to the infusion pump, wherein the smart device is configured to measure at least one of a flow rate and a pressure of a fluid in the IV line, and wherein the one or more processors are further programmed and/or configured to determine, based on the measured noise or vibration of the infusion pump and the at least one of the flow rate and the pressure of the fluid in the IV line, an unintended flow of the fluid in the IV line.

Clause 35. The system of any of clauses 21-34, wherein the one or more processors are further programmed and/or configured to determine, based on the measured noise or vibration of the infusion pump, a migration or a dislodgement of an IV line connected to the infusion pump.

Clause 36. The system of any of clauses 21-35, wherein the housing further includes: a user input component configured to receive a user input from a user.

Clause 37. The system of any of clauses 21-36, wherein the housing further includes a user feedback component configured to provide feedback to the user.

Clause 38. The system of any of clauses 21-37, further comprising: a smart device connected to an IV line connected to the infusion pump, wherein the user feedback component is configured to produce a same type of visual output as the smart device based on a communication connection established between the wireless communication device and the smart device.

Clause 39. The system of any of clauses 21-38, wherein the housing further includes an identifier element encapsulating a unique identifier associated with the peripheral device, and wherein the identifier element includes at least one of the following: a barcode, an RFID tag, or any combination thereof.

Clause 40. The system of any of clauses 21-39, further comprising: the infusion pump, wherein the peripheral device is connected to the infusion pump.

Clause 41. The system of any of clauses 21-40, further comprising: the external device, wherein in the external device includes the one or more processors; and a smart device connected to an IV line connected to the infusion pump, wherein the smart device is configured to communicate, to the external device, information associated with a fluid in the IV line.

Clause 42. The system of any of clauses 21-41, wherein the peripheral device is a reusable device configured to be at least one of: (i) connected to at least one different infusion pump after use with the infusion pump and (ii) connected to the infusion pump during fluid delivery to a plurality of different patients.

Clause 43. The system of claim any of clauses 21-42, wherein the smart device is a disposable device configured to be disposed of after use with the IV line.

Clause 44. A peripheral device for a medical device, comprising: a housing including: a user feedback component configured to provide feedback to the user, and a wireless communication device configured to communicate with a smart device connected to an IV line, wherein the user feedback component is configured to produce a same type of visual output as the smart device based on a communication connection established between the wireless communication device and the smart device; and a mechanical interface configured to connect the housing to an exterior surface of the medical device.

Clause 45. The peripheral device of clause 44, wherein a portion of the housing is connected directly to the IV line.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, can be implemented;

FIGS. 2A-2G are diagrams of non-limiting embodiments or aspects of one or more systems and/or one or more devices of FIG. 1;

FIG. 3 is a diagram of non-limiting embodiments or aspects of components of one or more devices and/or one or more systems of FIGS. 1 and 2A-2G;

FIG. 4A is a side view of non-limiting embodiments or aspects of an implementation of a needleless connector;

FIG. 4B is a side view of non-limiting embodiments or aspects of an implementation of a smart device and a needleless connector;

FIG. 4C is a side view of non-limiting embodiments or aspects of an implementation of a smart device and a needleless connector;

FIG. 5A is a perspective view of non-limiting embodiments or aspects of an implementation of a smart device and a needleless connector;

FIG. 5B is a top view of non-limiting embodiments or aspects of an implementation of a smart device and a needleless connector;

FIG. 5C is a graph of non-limiting embodiments or aspects of a force signal over time;

FIGS. 6A and 6B show non-limiting embodiments or aspects of output of one or more systems and/or one or more devices of FIG. 1;

FIG. 7 is a diagram of non-limiting embodiments or aspects of an implementation of a smart device for detecting an extravasation and/or an infiltration of a medication in a catheter;

FIG. 8 is a flowchart of non-limiting embodiments or aspects of a process for identifying a lumen;

FIG. 9 is a flowchart of non-limiting embodiments or aspects of a process for identifying a lumen;

FIG. 10 is a flowchart of non-limiting embodiments or aspects of a process for determining a tubing or IV line change;

FIG. 11 is a flowchart of non-limiting embodiments or aspects of a process for event monitoring; and

Appendix A includes details regarding non-limiting embodiments or aspects of devices, methods, systems, and computer program products described herein.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

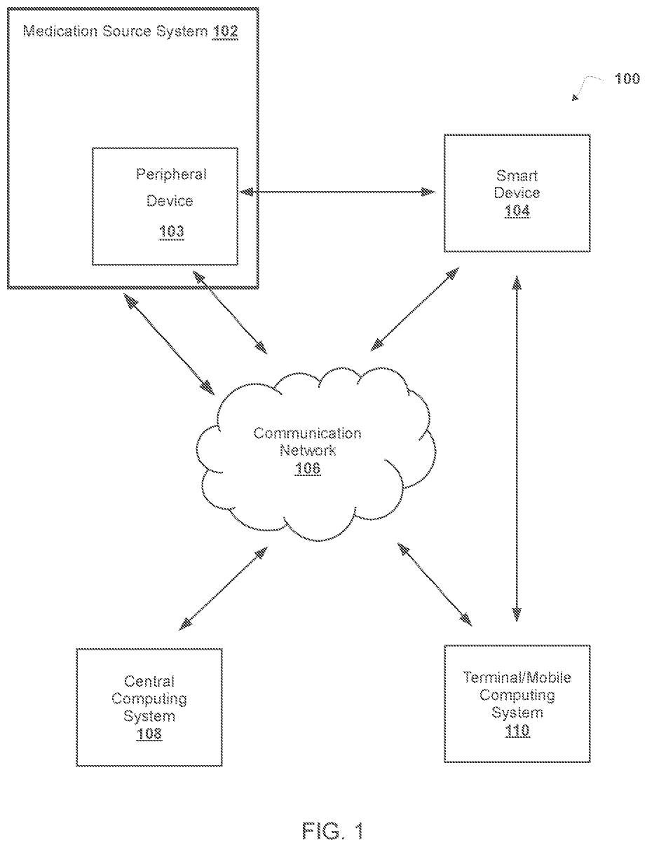

Referring now to FIG. 1, FIG. 1 is a diagram of an example environment 100 in which devices, systems, methods, and/or products described herein, may be implemented. As shown in FIG. 1, environment 100 includes medication source system 102, peripheral device 103, smart device 104, communication network 106, central computing system 108, and terminal/mobile computing system 110. Systems and/or devices of environment 100 can interconnect via wired connections, wireless connections, or a combination of wired and wireless connections.

In some non-limiting embodiments or aspects, medication source system 102 includes one or more devices capable of and/or associated with delivering one or more fluids to one or more lumens (e.g., to fluid lines, to IV lines, etc.). For example, medication source system 102 may include one or more manual fluid delivery systems (e.g., one or more IV bags, one or more syringes, etc.), an infusion pump system including one or more infusion pumps, another durable medical device associated with a fluid delivery to a patient (e.g., a medication dispensing cabinet, an ultrasound device, a sharps collector, etc.), and/or the like. In some non-limiting embodiments, peripheral device 103 may include a plurality of peripheral devices 103 (e.g., one or more other and/or different types of peripheral device 103, etc.). In some non-limiting embodiments, smart device 104 may include a plurality of smart devices 104 (e.g., one or more other and/or different types of smart devices 104, etc.).

In some non-limiting embodiments or aspects, medication source system 102 includes one or more devices capable of receiving information and/or data from peripheral device 103, smart device 104, communication network 106, central computing system 108, and/or terminal/mobile computing system 110 and/or communicating information and/or data to peripheral device 103, smart device 104, communication network 106, central computing system 108, and/or terminal/mobile computing system 110. For example, medication source system 102 may include one or more computing systems including one or more processors (e.g., one or more computing devices, one or more mobile computing devices, etc.).

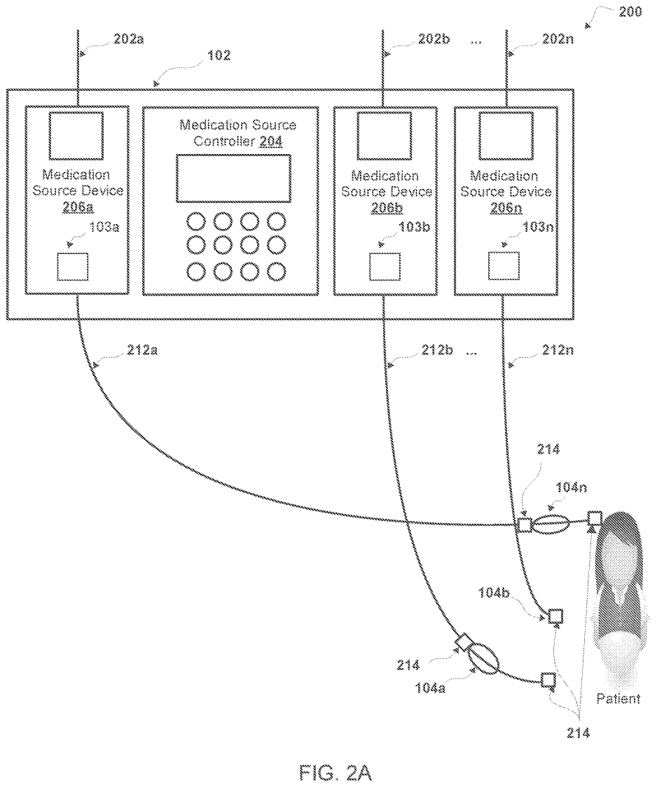

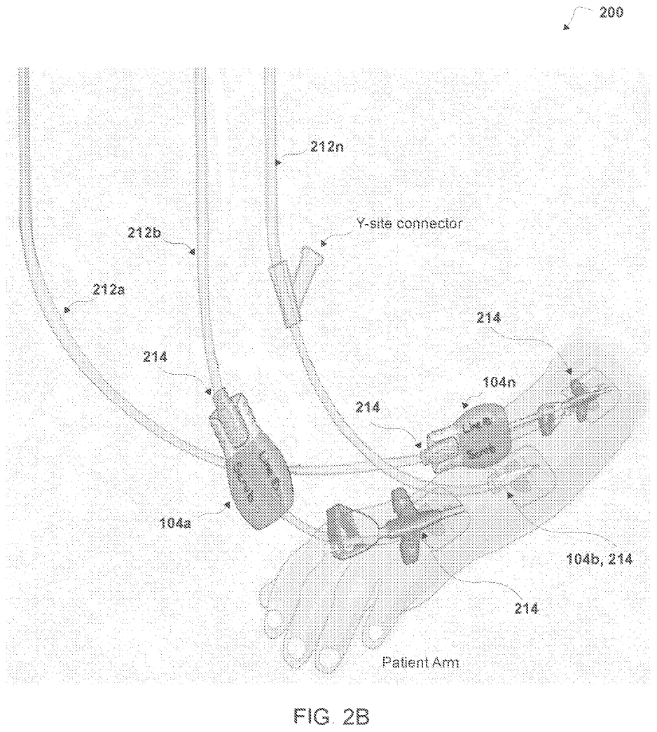

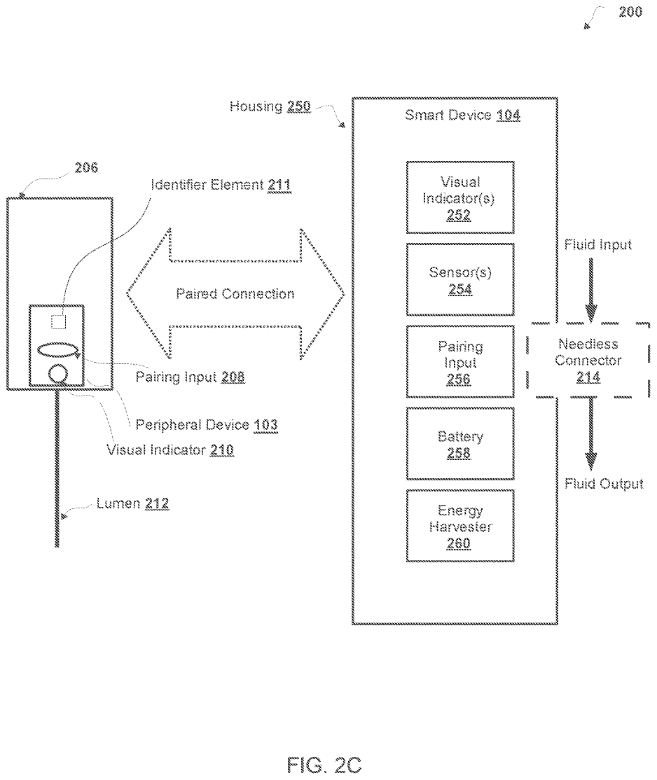

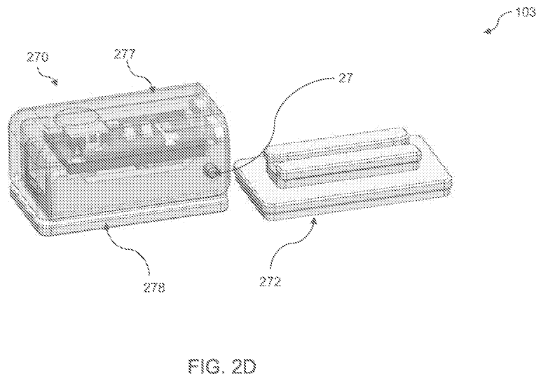

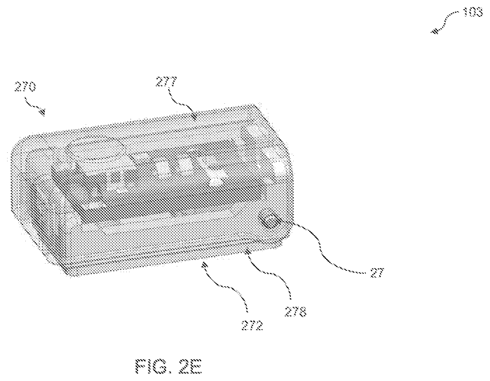

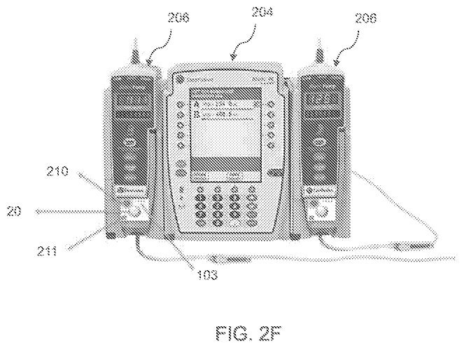

Further details regarding non-limiting embodiments or aspects of medication source system 102 are provided below with regard to FIGS. 2A and 2F.

In some non-limiting embodiments or aspects, peripheral device 103 includes one or more devices capable of receiving information and/or data from medication source system 102, one or more other peripheral devices 103, smart device 104, communication network 106, central computing system 108, and/or terminal/mobile computing system 110 and/or communicating information and/or data to medication source system 102, one or more other peripheral devices 103, smart device 104, communication network 106, central computing system 108, and/or terminal/mobile computing system 110. For example, peripheral device 103 may include one or more computing systems including one or more processors (e.g., one or more computing devices, one or more mobile computing devices, etc.). In some non-limiting embodiments or aspects, peripheral device 103 may be capable of receiving information (e.g., from medication source system 102, from terminal/mobile computing system 110, from one or more other peripheral devices 103, from smart device 104, etc.) via a short range wireless communication connection (e.g., an NFC communication connection, an RFID communication connection, a Bluetooth® communication connection, and/or the like), and/or communicating information (e.g., to medication source system 102, to terminal/mobile computing system 110, to one or more other peripheral devices 103, to smart device 104, etc.) via a short range wireless communication connection.

In some non-limiting embodiments or aspects, peripheral device 103 is configured to be connected to a medication source device (e.g., an infusion pump, etc.), a patient bed (e.g., to tie a patient to a bed/floor/unit, etc.), and/or other capital equipment that may connect to an IV administration set and/or connected to an exterior of a medical device (e.g., a fluid line, an IV line, etc.) connected to a medication source device. As an example, peripheral device 103 may not include any fluid contact elements and/or may be located completely outside a fluid path of an IV administration set when connected to an infusion pump (and/or another device). Peripheral device 103 may be connected to an infusion pump (or another device) during manufacture of the infusion pump or after manufacture of the infusion pump and/or during use of the infusion pump on a per patient or per device (e.g., per pump, etc.) basis.

In some non-limiting embodiments or aspects, peripheral device 103 may be a reusable or durable device configured to be connected to a plurality of different devices (e.g., to a plurality of different durable medication source devices, etc.). For example, peripheral device 103 may be connected to a first medication source device (e.g., a first infusion pump, etc.) for a first period of time and connected to a second medication source device (e.g., a second infusion pump different than the first infusion pump, etc.) for a second period of time different than the first period of time. As an example, peripheral device 103 may be connected to a medication source device for a first period of time associated with a first patient and be connected to a same or different medication source device for a second period of time associated with a second patient different than the first patient. In such an example, smart device 104 may be a disposable device configured to be disposed of after use with a single device (e.g., a single fluid line, a single IV line, etc.) and/or a single patient.

Further details regarding non-limiting embodiments or aspects of peripheral device 103 are provided below with regard to FIGS. 2A and 2C-2G.

In some non-limiting embodiments or aspects, smart device 104 includes one or more devices capable of receiving information and/or data from medication source system 102, peripheral device 103, one or more other smart devices 104, communication network 106, central computing system 108, and/or terminal/mobile computing system 110 and/or communicating information and/or data to medication source system 102, peripheral device 103, one or more other smart devices 104, communication network 106, central computing system 108, and/or terminal/mobile computing system 110. For example, smart device 104 may include one or more computing systems including one or more processors (e.g., one or more computing devices, one or more mobile computing devices, etc.). In some non-limiting embodiments or aspects, smart device 104 may be capable of receiving information (e.g., from medication source system 102, from terminal/mobile computing system 110, from peripheral device 103, from one or more other smart devices 104, etc.) via a short range wireless communication connection (e.g., an NFC communication connection, an RFID communication connection, a Bluetooth® communication connection, and/or the like), and/or communicating information (e.g., to medication source system 102, to terminal/mobile computing system 110, to peripheral device 103, to one or more other smart devices 104, etc.) via a short range wireless communication connection.

Further details regarding non-limiting embodiments or aspects of smart device 104 are provided below with regard to FIGS. 2A-2C, 3, 4A-4C, 5A-5C, 6A, 6B, and 7.

In some non-limiting embodiments or aspects, communication network 106 includes one or more wired and/or wireless networks. For example, communication network 106 includes a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or any combination of these or other types of networks.

In some non-limiting embodiments or aspects, central computing system 108 includes one or more devices capable of receiving information and/or data from medication source system 102, peripheral device 103, smart device 104, communication network 106, and/or terminal/mobile computing system 110 and/or communicating information and/or data to medication source system 102, peripheral device 103, smart device 104, communication network 106, and/or terminal/mobile computing system 110. For example, medication source system 102 may include one or more computing systems including one or more processors (e.g., one or more computing devices, one or more mobile computing devices, etc.). In some non-limiting embodiments or aspects, central computing system 108 includes a secure hospital server and/or one or more secure hospital databases that store personally identifiable information (PII) and/or Health Insurance Portability and Accountability Act (HIPAA) protected information.

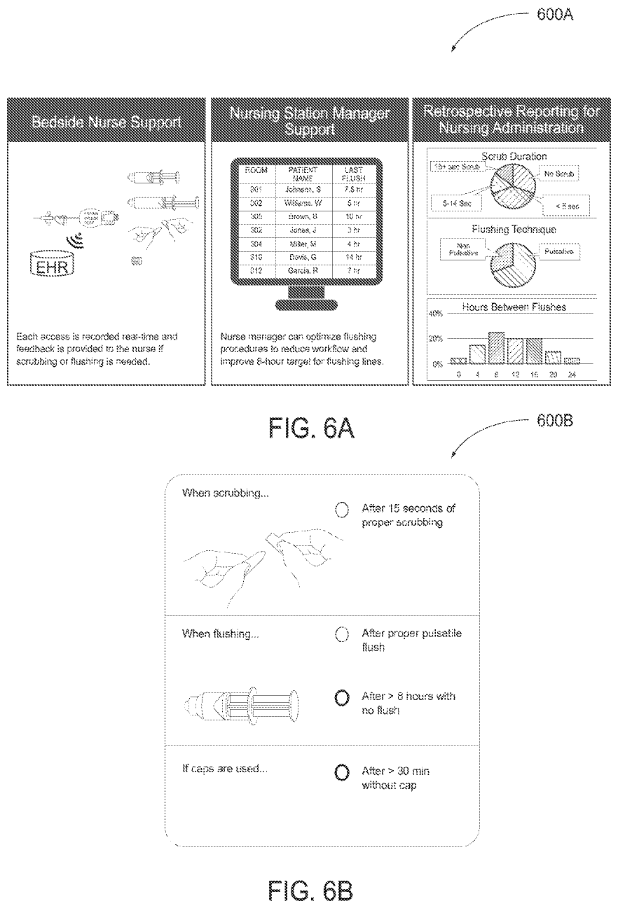

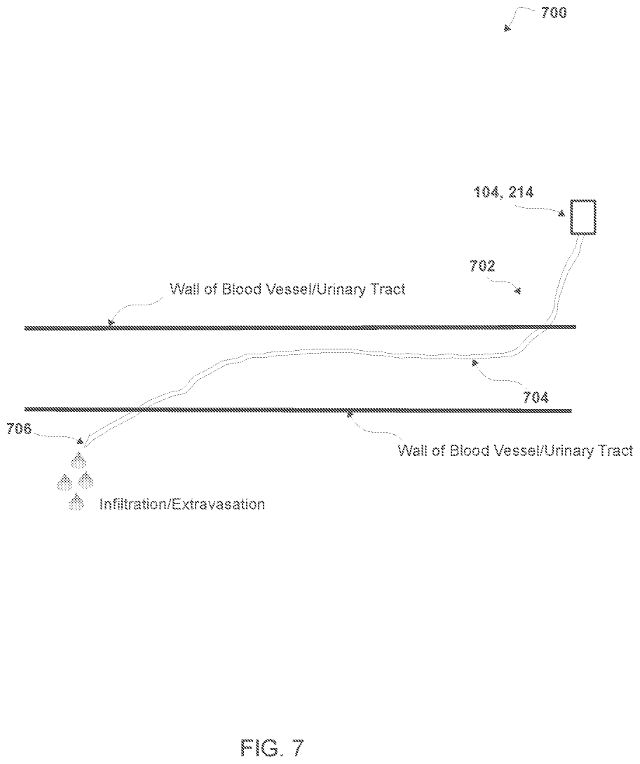

In some non-limiting embodiments or aspects, terminal/mobile computing system 110 includes one or more devices capable of receiving information and/or data from medication source system 102, smart device 104, communication network 106, and/or central computing system 108 and/or communicating information and/or data to medication source system 102, peripheral device 103, smart device 104, communication network 106, and/or central computing system 108. For example, terminal/mobile computing system 110 may include one or more computing systems including one or more processors (e.g., one or more computing devices, one or more mobile computing devices, etc.). In some non-limiting embodiments or aspects, terminal/mobile computing system 110 includes a nurse station in a hospital. For example, as shown in an implementation 600A FIG. 6A, terminal/mobile computing system 110 may provide bedside nurse support (e.g., recordation of each access to needleless connector 214 and/or lumen 212 in real-time, recordation of replacement of a lumen or IV line, and feedback to a nurse if scrubbing or flushing is determined to be due or needed according to the recorded access, if unintended flow is occurring in a lumen or IV line, if catheter migration or dislodgement has occurred, etc.), nursing station manager support (e.g., optimization of flushing procedures to reduce workflow and improve timed targets for flushing a needleless connector 214 and/or lumen 212, etc.), retrospective reporting for nursing administration (e.g., a scrub duration, a flushing technique, a time between flushes, and/or the like for a needleless connector 214 and/or lumen 212, etc.), and/or the like.

Referring now to FIGS. 2A-2G, FIGS. 2A-2G are diagrams of non-limiting embodiments or aspects of one or more systems and/or one or more devices of FIG. 1. As shown in FIGS. 2A, 2C, and 2F, medication source system 102 may include a medication source controller 204 and/or one or more medication source devices 206 (e.g., a plurality of mediation source devices 206a, 206b, . . . 206n, etc.). As an example, medication source controller 204 may include an infusion pump controller and/or medication source device 206 may include an infusion pump. In such an example, medication source system 102 may include the BD Alaris™ system. For example, as shown in FIG. 2F, medication source system 102 may include a BD Alaris™ PC Unit and one or more BD Alaris™ Pump Modules. As another example, medication source controller 204 may include a bed-side console or computing device, which may be separate from an infusion pump system, and/or medication source device 206, which may be separate from an infusion pump, may be associated with and/or connected to a medication source (e.g., an IV bag, a syringe, an end of an IV line connected and proximal to an IV bag or a syringe, etc.).

As shown in FIG. 2A, the plurality of medication source devices 206a, 206b, . . . 206n may be connected to a plurality of lumens (e.g., fluid lines, etc.) 202a, 202b, . . . 202n (e.g., for receiving a fluid and/or a medication at medication source system 102) and/or a plurality of lumens (e.g., fluid lines, etc.) 212a, 212b, . . . 212n (e.g., for delivering a fluid and/or a medication from medication source system 102, etc.). As shown in FIGS. 2A, 2C and 2F, peripheral device 103 may be connected to medication source device 206 (e.g., an infusion pump, etc.), and peripheral device 103 may include pairing input 208 (e.g., a button, input component 310, etc.), user feedback device or visual indicator 210 (e.g., a multi-color LED(s), output component 312, etc.), and/or identifier element 211 encapsulating a unique identifier associated with the peripheral device (e.g., a barcode, an RFID tag, etc.). As shown in FIGS. 2A and 2B, the plurality of lumens 212a, 212b, . . . 212n may be connected to a plurality of smart devices 104a, 104b, . . . 104n.

However, non-limiting embodiments or aspects are not limited thereto and a patient may be connected to only a single lumen, and the lumen may (or may not) be connected to any medication source device 206 and/or fluid source. For example, if a patient is connected to a single lumen, a smart device 104 may be connected to the single lumen connected to a medication source device 206 with peripheral device 103 attached thereto, and the smart device 104 and/or peripheral device 103 may be associated with a particular color associated with that lumen. A doctor and/or a pharmacy may issue an order for a type of medication to be delivered to the patient, and the order for the type of medication can link the color of the lumen to the order to reduce or avoid medication delivery errors. As an example, a first patient may have a “pink” LED light illuminated on smart device 104 and/or peripheral device 103, and an order from a doctor and/or a pharmacy that includes a new type of medication or therapy to be delivered and instructions for a user (e.g., a nurse, etc.) to deliver the new type of medication on the one and only “pink” line to implement an error checking process. When the user (e.g., the nurse, etc.) initiates delivery of the medication to the patient, the user can ensure that smart device 104 and/or peripheral device 103 is emitting the pink color for an additional safety check to ensure that the user has the right patient for the right type of medication. If smart device 104 and/or peripheral device 103 is emitting a color of light other than the “pink” light (e.g., a “green” light or a “blue” light) and the order for the medication indicates a that the medication should only be delivered via a “pink” colored lumen, the user (e.g., the nurse, etc.) may initiation of delivery to the patient (e.g., not deliver the medication to the patient, etc.) because the user can determine that the order and the color does not match, which indicates that there may be an issue and/or an incompatibility with medication for the patient and/or the lumen. For example, if the patient is connected to a single lumen, the order can indicate to deliver give saline on the “pink” line and to deliver antibiotics on the same “pink” line, etc. However, if there is a drug incompatibility ordered, the order can alter the user that additional flushing is indicated for the “pink” line to ensure that the pink line properly flushed before subsequent medications that may be incompatible with medications or fluids previously delivered via the “pink” line are delivered. As an example, if there are two lumens connected to a single patient (e.g., a single “pink” line and a single “blue” line) an order may indicate to deliver saline via the “pink” lumen or line and deliver antibiotics via the “blue” lumen or line, and/or the like. Accordingly, in some non-limiting embodiments or aspects, smart device 104 and/or peripheral device 103 may simply be used for proper patient lumen identification and/or to direct a user to use an optimal lumen for delivery of a fluid to the patient, which may ensure the proper therapy is delivered to the optimal or proper lumen, thereby reducing scenarios associated with a wrong dose, a wrong patient, a wrong lumen, and/or the like with smart device 104 and/or peripheral device 103.

Peripheral device 103 may include a housing 270 and a mechanical interface 272. Housing 270 may include sensor 280, wireless communication device 282 (e.g., communication interface 314, etc.), processor 284 (e.g., processor 304, etc.), user input/feedback device 286 (e.g., pairing input 208, visual indicator 210, input component 310, output component 213, etc.), battery 288, energy harvester 290, and/or identifier element 211. Mechanical interface 272 may be configured to connect housing 270 to an exterior surface of a device, such as medication source device 206 (e.g., an infusion pump, etc.), a bed, and/or the like and/or to an exterior of a medical device (e.g., a fluid line, an IV line, etc.) connected to medication source device 206. Additionally or alternatively, although not shown in FIGS. 2A-2G, a portion of housing 270 (e.g., a portion of housing 270 including sensor 280, etc.) may be attached directly to (e.g., clamped to via a clamp, etc.) lumen 212 connected to medication source device 206 at a location adjacent to medication source device 206 (e.g., at a portion of lumen 212 connected to a fluid output of medication source device 206, etc.).

As shown in FIGS. 2D and 2E, housing 270 may include cover 277 and base 278. Cover 277 may be rotatable about pin 279 connecting cover 277 to base 278. For example, cover 277 may rotate between a position in which other components of housing 270 (e.g., sensor 280, wireless communication device 282, processor 284, user input/feedback device 286. battery 288, energy harvester 290, a PCBA carrying these components, etc.) are covered by cover 277 (e.g., sealed from the environment by a combination of cover 277 and base 278, etc.), and a position in which the other components of housing 270 are not covered by cover 277 (e.g., accessible to a user, not sealed from the environment by a combination of cover 277 and base 278, etc.). As an example, cover 277 may be formed of a clear or transparent material that pivots about pin 279, for example, in response to a user actuating or pressing on pin 279 to activate a tactile switch on an underside of the PCBA.

In some non-limiting embodiments or aspects, housing 270 is removably connected to mechanical interface 272. For example, as shown in FIG. 2D, housing 270 may be connected to a separate mechanical interface 272 via an adhesive layer, a snap fit connection, and/or the like. In such an example, mechanical interface 272 may include a base plate configured to be connected to an exterior surface of infusion pump 206. In some non-limiting embodiments or aspects, housing 270 is integrated with mechanical interface 272. For example, as shown in FIG. 2E, a bottom of base 278 of housing 270 may include mechanical interface 272. In such examples, mechanical interface 272 may include an adhesive layer and/or a snap fit connector configured to form an adhesive and/or a snap fit connection to infusion pump 206.

Sensor 280 may be configured to measure a parameter associated with an infusion pump and/or a lumen or IV line connected to the infusion pump. For example, sensor 280 may include at least one of the following: a button configured for mechanical interaction with a door of an infusion pump, a piezoelectric sensor, a tilt switch, an accelerometer, an acoustic or vibration sensor, a force sensor (e.g., one or more piezoelectric elements or transducers, one or more force sensitive resistive (FSR) sensors, one or more strain gauges, etc.), a pressure sensor, an acoustic sensor, an optical sensor (e.g., an optical sensor configured to detect at least one of a color signature and a reflectance of a lumen 212 and/or a fluid flowing in lumen 212, etc.), an identification sensor (e.g., an identification sensor configured to detect an identification tag on a medical device connected to or being connected to peripheral device 103, such as a magnetometer configured to detect a magnetic material, a barcode scanner configured to read a bar code, etc.), a position sensor (e.g., a position sensor configured to detect movement of peripheral device 103, etc.); an RBG color sensor, or any combination thereof. In some non-limiting embodiments or aspects, peripheral device 103 (e.g., housing 270 of peripheral device 103, etc.) may not include sensor 280.

In some non-limiting embodiments or aspects, sensor 280 may be configured to measure door opening and/or closing events of a door of infusion pump 206 that provides access to a lumen or IV line connected to infusion pump 206. As an example, a button on peripheral device 103 may be pushed to activate a switch on a front-plate of infusion pump 206 to open a door of infusion pump 206. As an example, a piezoelectric sensor or other sensor within peripheral device 103 may sense when the door is opened. As an example, one or more tilt switches may be used to sense a state of the door. As an example, information associated with the state of the door may be obtained via digital communication with the infusion pump 206. Accordingly, sensor 280 may provide information and/or data associated with when tubing or IV line changes for infusion pump 206 are performed. In this way, improved documentation of tubing or IV line changes may be captured passively. In some non-limiting embodiments or aspects, peripheral device 103 may interface and/or communicate with central computing system 108 and/or terminal/mobile computing system 110 to provide reminders to clinicians regarding when tubing changes are due.

In some non-limiting embodiments or aspects, sensor 280 includes an acoustic or vibration sensor configured to measure a noise or vibration of infusion pump 206. For example, peripheral device 103 may be configured verify a pumping and/or flow status of infusion pump 206 (e.g. on/off, etc.) by measuring up vibration and/or sound signatures of mechanisms and/or operations of infusion pump 206. In some non-limiting embodiments or aspects, peripheral device 103, when paired with an in-line smart device 104 including a flow and/or a pressure sensor, may be configured to verify if unintended flow is occurring in the lumen or IV line attached to infusion pump 206 (e.g., flow or pressure sensed without pumping action detected, etc.). Further, peripheral device 103 can be configured to measure the vibration and/or sound signature of infusion pump 206 (and/or another device, such as the lumen or IV line, and/or the like) to account for the vibration and/or sound signature of the infusion pump when detecting a vibration and/or sound signature associated with other elements (e.g., for detection of migration of the catheter or dislodgement of the catheter, etc.).

Wireless communication device 282 may be configured to communicate, to an external device (e.g., smart device 104, central computing system 108, terminal/mobile computing system 110, etc.), information associated with the measured parameter of infusion pump 206. For example, information associated with the measured parameter of infusion pump 206 may include at least one of the following: the measured parameter, a state of the infusion pump (e.g., a door open state, a door closed state, a pump on state, a pump off state, etc.), a time of a tube or IV line change, an indication of an unintended flow, an indication of a catheter migration or dislodgement, a unique identifier of peripheral device 103 and/or the infusion pump, or any combination thereof.

Processor 284 may be programmed and/or configured to determine, based on the measured parameter of infusion pump 206, a state of infusion pump 206. In some non-limiting embodiments or aspects, processor 284 may include a low power microcontroller unit (MCU).

In some non-limiting embodiments or aspects, processor 284 (and/or smart device 104, central computing system 108, terminal/mobile computing system 110, etc.) may be programmed and/or configured to determine, based on the measured parameter from sensor 280, a door open state and/or a door closed state of the door of infusion pump 206. For example, processor 284 (and/or smart device 104, central computing system 108, terminal/mobile computing system 110, etc.) may be programmed and/or configured to determine, based on the door open state and/or the door closed state of the door of the infusion pump (e.g., based on a change over time therein, etc.), that a tubing or IV line change for infusion pump 206 has been performed. As an example, an opening of the door followed by a closing of the door may indicate that a tubing or IV line change for infusion pump 206 has been performed.

In some non-limiting embodiments or aspects, processor 284 (and/or smart device 104, central computing system 108, terminal/mobile computing system 110, etc.) may be programmed and/or configured to determine, based on the measured noise or vibration of infusion pump 206, a pump on state and/or a pump off state of infusion pump 206. For example, processor 284 (and/or smart device 104, central computing system 108, terminal/mobile computing system 110, etc.) may be programmed and/or configured to determine, based on the pump on state and/or the pump off state, that unintended flow is occurring in the lumen or IV line attached to infusion pump 206 and/or that migration or dislodgement of the catheter has occurred. As an example, processor 284 (and/or smart device 104, central computing system 108, terminal/mobile computing system 110, etc.) may compare the measured noise or vibration of infusion pump 206 to one or more reference noise or vibration signatures associated with a pump on state, a pump off state, an unintended flow, a catheter migration or dislodgement, or any combination thereof, to determine the state of infusion pump 206 and/or one or more components connected thereto. For example, processor 284 (and/or smart device 104, central computing system 108, terminal/mobile computing system 110, etc.) may be programmed and/or configured to determine, based on the measured noise or vibration of the infusion pump, a migration or a dislodgement of an IV line connected to infusion pump 206. As an example, smart device 104 may be connected to the IV line connected to infusion pump 206, and smart device 104 may measure at least one of a flow rate and a pressure of a fluid in the IV line and provide the measured flow rate and/or pressure to processor 284 (and/or smart device 104, central computing system 108, terminal/mobile computing system 110, etc.). In such an example, processor 284 (and/or smart device 104, central computing system 108, terminal/mobile computing system 110, etc.) may be programmed and/or configured to determine, based on the measured noise or vibration of infusion pump 206 and the at least one of the flow rate and the pressure of the fluid in the IV line, an unintended flow of the fluid in the IV line.

User input/feedback device 286 may be configured to receive a user input from a user and/or to provide feedback to the user. For example, user input/feedback device 286 may include at least one of the following: a display, a light-emitting diode (LED), an audio output device (e.g., a buzzer, a speaker, etc.), or any combination thereof. In some non-limiting embodiments or aspects, user input/feedback device 286 includes pairing input 208 and/or visual indicator 210. In some non-limiting embodiments or aspects, visual indicator 210 may provide an indication to a clinician of what lumen or IV line an administration is flowing through, which may tie peripheral device 103 to one or more smart devices 104 as described herein. In this way, visual indicator 210 may associate infusion pump 206 with a specific administration set and/or catheter for which infusion pump 206 is utilized, as well as with a specific lumen or IV line on the administration set/catheter.

Battery 288 may include a rechargeable battery, a single use battery, a replaceable battery, or any combination thereof.

Energy Harvester 290 may include a thermoelectric energy harvester, a photovoltaic energy harvester, a piezoelectric energy harvester, and/or the like.

Battery 288 and/or energy harvester 290 may provide power for operating components of peripheral device 103, such as sensor 280, wireless communication device 282, processor 284, user input/feedback device 286, one or more components of device 300 included in peripheral device 103, and/or the like.

In some non-limiting embodiments or aspects, smart device 104 is configured to be removably connected to needleless connector 214 and/or a portion of lumen 212 proximate needleless connector 214, such as an IV lumen (e.g., a peripherally inserted central catheter (PICC), a peripheral intravenous catheter (PIVC), a central venous catheter (CVC), etc.), and/or the like. For example, smart device 104 may include a clamp, an adhesive, or other attachment means configured to removably connect smart device 104 to needleless connector 214 and/or lumen 212 proximate needleless connector 214. As an example, as shown in FIGS. 2A and 2B, smart device 104a may be connected to needleless connector 214 and/or a catheter lumen that connects a catheter to lumen 212b, and/or smart device 104n may be connected to needleless connector 214 and/or a catheter lumen that connects a catheter to lumen 212a. In some non-limiting embodiments or aspects, smart device 104 includes needleless connector 214. For example, smart device 104 may be integrated with needleless connector 214 (e.g., within a needleless connector 214 and/or within a catheter hub of a needleless connector of a fluid invasive device, etc.). As an example, as shown in FIGS. 2A and 2B, smart device 104b may include needleless connector 214 and/or a catheter hub that connects a catheter lumen to lumen 212n via a Y-site connector. In such an example, smart device 104 may include needleless connector 214 including housing 402 of needleless connector 214 within housing 250 (e.g., integrated with housing 250, encompassed within housing 250, etc.).

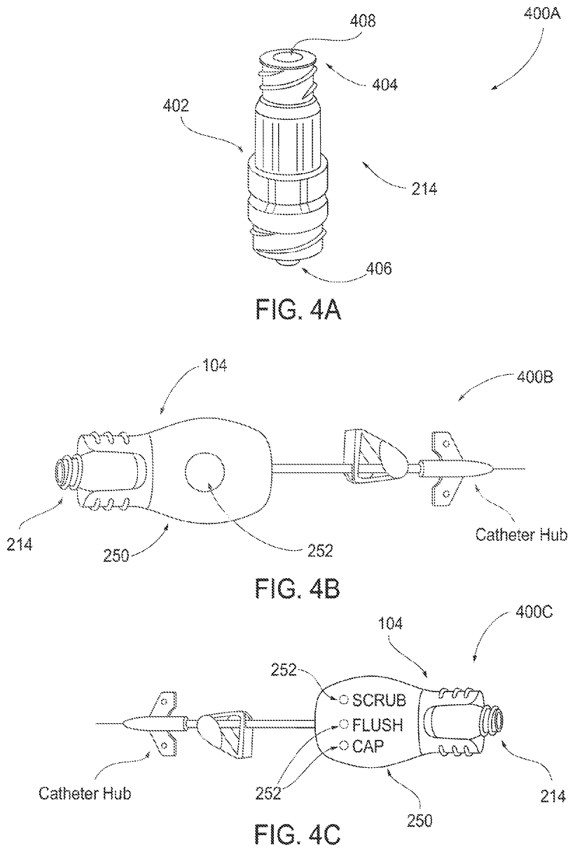

Referring also to FIG. 4A, FIG. 4A is a side view of non-limiting embodiments or aspects of an implementation 400A of a needleless connector 214. As shown in FIG. 4A, a needleless connector 214 may include a fluid flow path in a housing 402 between an inlet 404 and an outlet 406 opposite the inlet 404. Inlet 404 may be fluidically sealed by a displaceable septum 408 configured to be displaced to open or connect inlet 404 to the fluid flow path in response to connection of needleless connector 214 to a medical device (e.g., an infusion pump, an IV bag, a syringe, an IV line, etc.). For example, the needleless connector 214 may include the BD MaxPlus™ connector, the BD MaxZero™ needle-free connector, and/or the like. However, non-limiting embodiments or aspects are not limited thereto, and the needleless connector 214 may include any needleless connector 214 for use in fluid administration. In some non-limiting embodiments or aspects, one or more components of smart device 104 may be included within housing 402 of needleless connector 214. For example, housing 402 of needleless connector 214 may include housing 250 of smart device 104 (e.g., housing 250 may be integrated with housing 402, encompassed within housing 402, etc.).

As shown in FIG. 2C, smart device 104 may include visual indicator 252 (e.g., one or more visual indictors, a plurality of visual indicators, a multi-color LED(s), a plurality of LEDs, output component 312, etc.), sensor 254 (e.g., one or more sensors, a plurality of sensors, a sensor suite, etc.), pairing input 256 (e.g., one or more buttons, one or more force sensor, one or more accelerometers, input component 310, etc.), battery 258, and/or energy harvester 260 (e.g., a thermoelectric energy harvester, a photovoltaic energy harvester, a piezoelectric energy harvester, etc.). Visual indicator 252, sensor 254, pairing input 256, battery 258, energy harvester 260 and all or a portion of needleless connector 214 may be included within housing 250 of smart device 104. Visual indicator 252 may be visible through and/or extend from a sidewall of housing 250. Battery 258 and/or energy harvester 260 may provide power for operating components of smart device 104, such as visual indicator 252, sensor 254, pairing input 256, a rechargeable battery of battery 258, one or more components of device 300 included in smart device 104, and/or the like.

In some non-limiting embodiments or aspects, smart device 104 may include a label (e.g., a human readable label, etc.) that characterizes visual indicator 252 of smart device 104. For example, as shown in implementation 400C in FIG. 4C, smart device 104 may include labels associated with visual indicators 252 (e.g., on a sidewall of housing 250, etc.) that characterize each visual indicator 252 as configured for providing an indication associated a particular event, such as one of: a scrubbing event in which needleless connector 214 is scrubbed with a disinfectant (e.g., a label “SCRUB”, etc.); a flushing event in which needleless connector 214 is flushed with a solution (e.g., a label “FLUSH”, etc.); a connection or capping event in which needleless connector 214 is connected to a medical device (e.g., a label “CAP”, etc.); and/or the like. In some non-limiting embodiments or aspects, smart device 104 may include a single visual indicator 252 (e.g., as shown in implementation 400B in FIG. 4B). For example, smart device 104 may control single visual indicator 252 to illuminate in a particular color and/or in a particular pattern to provide an indication or prompt to a user, such as to illuminate a continuous green in response to sensing that scrubbing of needleless connector 214 has occurred for a predetermined period of time (e.g., 15 seconds, etc.), to illuminate a pulsating green in response to sensing that a proper pulsatile flush has occurred, to illuminate a pulsating red in response to determining that a pulsatile flush of needleless connector 214 has not occurred for a predetermined period of time (e.g., 88 hours, etc.), to illuminate a continuous red in response to determining that needleless connector 214 has not been capped with a disinfectant cap for a predetermined period of time (e.g., over minutes, etc.)

In some non-limiting embodiments or aspects, communication circuitry (e.g., communication interface 314, etc.) of peripheral device 103 is configured to establish communication with communication circuitry (e.g., communication interface 314, etc.) of smart device 104 based on user input to pairing input 208 of peripheral device 103 and user input to pairing input 256 of smart device 104. For example, peripheral device 103 may establish a short range wireless communication connection (e.g., an NFC communication connection, an RFID communication connection, a Bluetooth® communication connection, etc.) with smart device 104. As an example, visual indicator 210 may be configured to emit a predetermined light pattern (e.g., to blink rapidly to indicate that peripheral device 103 is in a pairing mode, etc.) in response to a predetermined user input to pairing input 208 (e.g., in response to a user pressing and holding a button of pairing input 208, etc.) of peripheral device 103. In such an example, smart device 104 may be configured to establish communication with peripheral device 103 (e.g., pair and/or activate a pairing sequence for pairing smart device 104 with peripheral device 103, etc.) in response to a predetermined user input to pairing input 256 (e.g., in response to a user pressing and holding a button of pairing input 256, etc.) of smart device 104 at a same time that peripheral device 103 is in the pairing mode.

In some non-limiting embodiments or aspects, when peripheral device 103 is paired with smart device 104, visual indicator 210 of peripheral device 103 and visual indicator 252 of smart device 104 are configured to provide a same type of visual output (e.g., a same color of light from a multi-colored LED, a same pattern of light, etc.) and/or to communicate information associated therewith to terminal mobile computing system 110 (e.g., to a nursing tablet or station, etc.). For example, and referring again to FIG. 2A, peripheral device 103a may be paired with smart device 104n and each of peripheral device 103a and smart device 104n may output a first color of light (e.g., red light), peripheral device 103b may be paired with smart device 104a and each of peripheral device 103b and smart device 104a may output a second color of light (e.g., green light), peripheral device 103n may be paired with smart device 104b and each of peripheral device 103n and smart device 104b may output a third color of light (e.g., blue light), and/or the like.

In some non-limiting embodiments or aspects, sensor 254 includes at least one of: one or more force sensors (e.g., one or more piezoelectric elements or transducers, one or more force sensitive resistive (FSR) sensors, one or more strain gauges, etc.); one or more pressure sensors; one or more acoustic sensors; one or more optical sensors (e.g., an optical sensor configured to detect at least one of a color signature and a reflectance of a medical device connected to smart device 104, etc.), one or more identification sensors (e.g., an identification sensor configured to detect an identification tag on a medical device connected to or being connected to the needleless connector 214, such as a magnetometer configured to detect a magnetic material, a barcode scanner configured to read a bar code, etc.); one or more position sensors (e.g., a position sensor configured to detect movement of smart device 104, etc.); one or more RBG color sensors; or any combination thereof.

The number and arrangement of systems, devices, and networks shown in FIGS. 1 and 2A-2G are provided as an example. There can be additional systems, devices and/or networks, fewer systems, devices, and/or networks, different systems, devices, and/or networks, or differently arranged systems, devices, and/or networks than those shown in FIGS. 1 and 2A-2G. Furthermore, two or more systems or devices shown in FIGS. 1 and 2A-G can be implemented within a single system or a single device, or a single system or a single device shown in FIGS. 1 and 2A-2G can be implemented as multiple, distributed systems or devices. Additionally, or alternatively, a set of systems or a set of devices (e.g., one or more systems, one or more devices, etc.) of environment 100 can perform one or more functions described as being performed by another set of systems or another set of devices of environment 100.

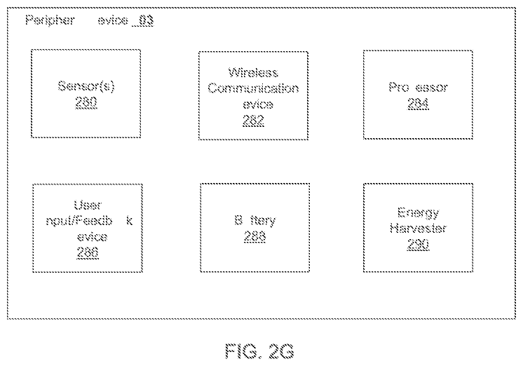

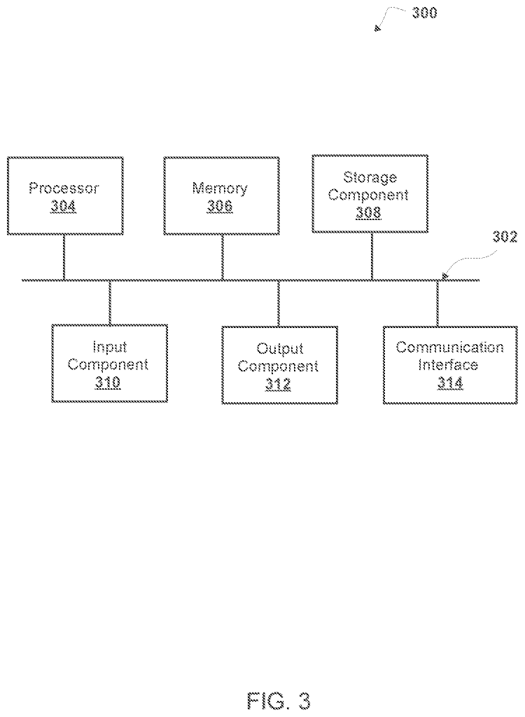

Referring now to FIG. 3, FIG. 3 is a diagram of example components of a device 300. Device 300 may correspond to one or more devices of medication source system 102, peripheral device 103, smart device 104, and/or one or more devices of communication network 106, one or more devices of central computing system 108, one or more devices of terminal/mobile computing system 110, one or more devices of medication source controller 204, and/or one or more devices of medication source device 206. In some non-limiting embodiments or aspects, one or more devices of medication source system 102, peripheral device 103, smart device 104, and/or one or more devices of communication network 106, one or more devices of central computing system 108, one or more devices of terminal/mobile computing system 110, one or more devices of medication source controller 204, and/or one or more devices of medication source device 206 can include at least one device 300 and/or at least one component of device 300. As shown in FIG. 3, device 300 may include a bus 302, a processor 304, memory 306, a storage component 308, an input component 310, an output component 312, and a communication interface 314.

Bus 302 may include a component that permits communication among the components of device 300. In some non-limiting embodiments or aspects, processor 304 may be implemented in hardware, firmware, or a combination of hardware and software. For example, processor 304 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), a microcontroller (MCU), etc.) that can be programmed to perform a function. Memory 306 may include random access memory (RAM), read only memory (ROM), and/or another type of dynamic or static storage device (e.g., flash memory, magnetic memory, optical memory, etc.) that stores information and/or instructions for use by processor 304.

Storage component 308 may store information and/or software related to the operation and use of device 300. For example, storage component 308 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 310 may include a component that permits device 300 to receive information, such as via user input (e.g., a touch screen display, a keyboard, a keypad, a mouse, a button, a switch, a microphone, a camera, an electroencephalogram (EEG) monitor, etc.). Additionally, or alternatively, input component 310 may include a sensor for sensing information (e.g., a global positioning system (GPS) component, an accelerometer, a gyroscope, an actuator, etc.). Output component 312 may include a component that provides output information from device 300 (e.g., a display, a speaker, one or more light-emitting diodes (LEDs), and/or the like).

Communication interface 314 may include a transceiver-like component (e.g., a transceiver, a separate receiver and transmitter, etc.) that enables device 300 to communicate with other devices, such as via a wired connection, a wireless connection, or a combination of wired and wireless connections. Communication interface 314 may permit device 300 to receive information from another device and/or provide information to another device. For example, communication interface 314 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 300 may perform one or more processes described herein. Device 300 may perform these processes based on processor 304 executing software instructions stored by a computer-readable medium, such as memory 306 and/or storage component 308. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 306 and/or storage component 308 from another computer-readable medium or from another device via communication interface 314. When executed, software instructions stored in memory 306 and/or storage component 308 may cause processor 304 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 306 and/or storage component 308 may include data storage or one or more data structures (e.g., a database, etc.). Device 300 may be capable of receiving information from, storing information in, communicating information to, or searching information stored in the data storage or one or more data structures in memory 306 and/or storage component 308. For example, the information may input data, output data, medical data, or any combination thereof.

The number and arrangement of components shown in FIG. 3 are provided as an example. In some non-limiting embodiments or aspects, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

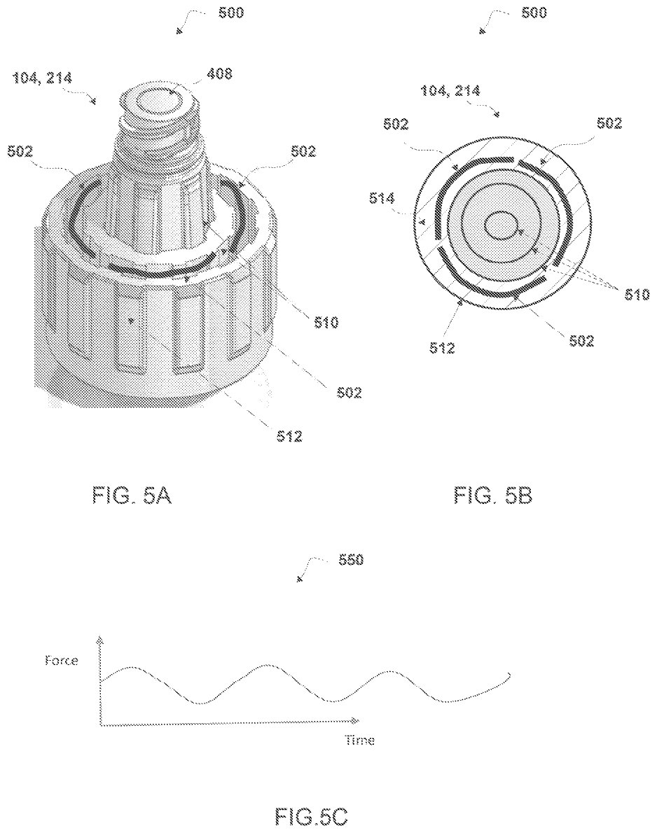

FIG. 5A is a perspective view and FIG. 5B is a top view of non-limiting embodiments or aspects of an implementation 500 of smart device 104 including needleless connector 214. Referring also to FIG. 4A, needleless connector 214 may include a fluid flow path in housing 402 between inlet 404 and outlet 406 opposite the inlet 404. Inlet 404 may be fluidically sealed by displaceable septum 408 configured to be displaced to open or connect inlet 404 to the fluid flow path in response to connection of needleless connector 214 to a medical device (e.g., an infusion pump, an IV bag, a syringe, an IV line, etc.). Referring again to FIGS. 5A and 5B, in some non-limiting embodiments, smart device 104 may include sensor 254. For example, sensor 254 may include force sensor 502 connected to needleless connector 214. As an example, force sensor 502 may be configured to sense, detect, and/or determine a force signal. In such an example, at least one of: a scrubbing event in which the needleless connector is scrubbed with a disinfectant, a flushing event in which the needleless connector is flushed with a solution, a connection event in which the needleless connector is connected to a medical device, or any combination thereof, may be determined based on the force signal (e.g., by smart device 104, etc.). In such an example, a pattern of events including a plurality of the least one of: the scrubbing event in which the needleless connector is scrubbed with the disinfectant, the flushing event in which the needleless connector is flushed with the solution, the connection event in which the needleless connector is connected to the medical device, a time between one or more detected events, or any combination thereof may be determined based on the force signal, and a medication administration event in which a medication is administered to a patient via needleless connector 214 may be determined based on the pattern of events. As an example, a standard medical practice may assume a Scrub-Flush-Scrub-MedAdmin-Scrub-Flush-Scrub pattern or sequence of events and, therefore, detection of three access of luer connectors may be interpreted by smart device 104 as a medication administration event. For example, FIG. 5C is a graph 550 of non-limiting embodiments or aspects of a force measurement or signal over time. As shown in FIG. FIG. 5C, pulsatile flushing may be determined or detected by force measurement, for example, when flushing is achieved by intermittent pressure pulses applied to a plunger of a flush syringe, and smart device 104 can detect occurrences of pulsatile flushes by identifying periodic force signals between x-y Hz in a force signal perpendicular to a surface of septum 408 of needleless connector 214. For example, smart device 104 may determine, based on the force signal indicating periodic forces in the second direction perpendicular to the surface of the septum facing in the first direction, the flushing event, and that the flushing event includes a pulsatile flushing event.

In some non-limiting embodiment or aspects, smart device may 104 may include communication circuitry configured to transmit the force signal to a remote computing system. For example, medication source system 102, central computing system 108, and/or terminal/mobile computing system 110 may obtain the force signal from smart device 104 and/or needleless connector 214 and process the force signal to determine at least one of: a scrubbing event in which the needleless connector is scrubbed with a disinfectant, a flushing event in which the needleless connector is flushed with a solution, a connection event in which the needleless connector is connected to a medical device, or any combination thereof.

In some non-limiting embodiments or aspects, force sensor 502 includes at least one of: a piezoelectric element, a force sensitive resistive (FSR) sensor, a strain gauge, or any combination thereof. In some non-limiting embodiments or aspects, force sensor 502 is positioned between an outer surface of inner wall 510 (e.g., an inner harder plastic wall) of needleless connector 214 defining the fluid flow path of needleless connector 214 and an inner surface of an outer wall 512 (e.g., a softer, a more flexible, a more pliable, a rubber, etc. wall) of needleless connector 214 surrounding the inner wall 510 of needleless connector 214. In some non-limiting embodiments or aspects, an area between an outer surface of inner wall 510 (e.g., an inner harder plastic wall) of needleless connector 214 defining the fluid flow path of needleless connector 214 and an inner surface of an outer wall 512 (e.g., a softer, a more flexible, more, a more pliable, a rubber, etc. wall) of needleless connector 214 surrounding the inner wall 510 of needleless connector 214, which may be held by a user during cleaning and/or connection to another medical device, may be filled with a rubber or other pliable type material 514 including force sensors 502 as force sensing films within the material 514 between the inner wall 510 and the outer wall 512. In some non-limiting embodiments or aspects, force sensors 502 may be located between inner wall 510 and outer wall 512 below threading on and/or proximal to inlet 404 of needleless connector 214.

In some non-limiting embodiments or aspects, force sensor 502 includes a plurality of force sensors 502 positioned around the fluid flow path of needleless connector 214 between the outer surface of inner wall 510 of needleless connector 214 defining the fluid flow path of needleless connector 214 and the inner surface of outer wall 512 of needleless connector 214 surrounding inner wall 510 of needleless connector 214. For example, inlet 404 of needleless connector 214 may include septum 408 including a surface facing in a first direction, and force sensor 502 may be configured to detect a force in a second direction perpendicular to the surface of the septum facing in the first direction. As an example, the flushing event, which may include a pulsatile flushing event, may be determined based on the force signal indicating periodic forces in the second direction perpendicular to the surface of the septum facing in the first direction.

In some non-limiting embodiments or aspects, sensor 254 includes a pressure sensor, and the pressure sensor is one of: in direct contact with a fluid in the fluid flow path of the needleless connector; located within an inner wall of the needleless connector defining the fluid flow path of the needleless connector, and located within a wall of a lumen connected to the needleless connector. For example, smart device 104 may determine or detect pulsatile flush, a flush, and or a med-administration by the pressure sensor in contact with the fluid path in the needleless connector 214 and/or a lumen thereof.

In some non-limiting embodiments or aspects, sensor 254 includes an optical sensor configured to detect at least one of a color signature and a reflectance of a medical device connected to and/or being connected to needleless connector 214, and smart device 104 may determine a type of the medical device based on the at least one of the color signature and the reflectance of the medical device. For example, a color signature and/or the reflectance of the medical device may be indicative of a syringe, an IV bag, an infusion pump, and/or a particular type thereof.

In some non-limiting embodiments or aspects, sensor 254 includes an identification sensor configured to detect an identification tag on a medical device connected to or being connected to the needleless connector. For example, the identification sensor may include a magnetometer, and the identification tag may include a magnetic material on and/or integrated with needleless connector 214.

In some non-limiting embodiments or aspects, sensor 254 includes a position sensor configured to detect movement of the needleless connector. For example, a movement of the patient, a fall event of the patient, a movement of a bed of the patient may be determined (e.g., by smart device 104, etc.) based on the detected movement of the needleless connector.

In some non-limiting embodiments or aspects, sensor 254 includes an RGB color sensor configured to detect a color of a fluid in the fluid flow path of the needleless connector. For example, at least one of a blood-draw in the needleless connector and a retention of blood in the needleless connector may be determined (e.g., by smart device 104, etc.) based on the color of the fluid detected in the fluid flow path of the needleless connector.

In some non-limiting embodiments or aspects, sensor 254 includes flow sensor and/or a pressure sensor configured to detect a flow rate and/or a pressure of a fluid in the lumen or IV line.

In some non-limiting embodiments or aspects, smart device 104 including needleless connector 214 may include visual indicator 252, and visual indicator 252 may be configured to provide a visual indication associated with the at least one of: the scrubbing event in which the needleless connector is scrubbed with the disinfectant, the flushing event in which the needleless connector is flushed with the solution, the connection event in which the needleless connector is connected to the medical device, or any combination thereof. For example, as shown in an implementation 600B in FIG. 6B, smart device 104 may provide direct patient-side feedback (e.g., via an LED light to a nurse, etc.) in response to (i) detecting that needleless connector 214 and/or lumen 212 thereof has not been scrubbed for a predetermined period of time and/or before a scheduled use, (ii) detecting that needleless connector 214 and/or lumen 212 thereof has not been scrubbed for a sufficient period of time prior to accessing a catheter line, (iii) detecting that a flush of needleless connector 214 and/or lumen 212 is due, (iv) detecting that a disinfection cap was not attached after a previous access to needleless connector 214 and/or lumen 212, and/or the like. For example, smart device 104 may include needleless connector 214, and needleless connector 214 may be configured to detect at least one of a scrubbing event, a flushing event, a connection or capping event, or any combination thereof. As an example, and needleless connector 214 may be configured to provide information and/or data associated with a detected scrubbing event, a detected flushing event, and/or a detected connection or capping event (e.g., with processor 304, memory 306, storage component 308, input component 310, output component 312, etc.) to store events and report compliance performance for compliance event monitoring.

FIG. 7 is a diagram of non-limiting embodiments or aspects of an implementation 700 of a smart device for detecting an extravasation or an infiltration of a medication in a catheter. As shown in FIG. 7, smart device 104 may be connected to or integrated with a needleless connector 214 at a catheter hub of catheter 702 including a catheter lumen or line 704 and a needle tip 706 for delivering fluid to a patient at an opposite end of the catheter line 704 from smart device 104. Catheter 702 may be inserted in a blood vessel (e.g., a vein, an artery, etc.) or a urinary tract of the patient. For example, the location of the tip 706 of the needle may be within the blood vessel or the urinary tract of the patient, within a wall of the blood vessel or a wall of the urinary tract of the patient, or outside the blood vessel or the urinary tract and the wall of the blood vessel or the wall of the urinary tract of the patient. In some non-limiting embodiments or aspects, smart device 104 including catheter 702 may include a wired and/or a wireless transmitted configured to (e.g., via a wire, wirelessly, etc.) transmit the at least one signal (and/or a variation in the at least one signal over a period of time, a location of the tip of the needle with respect to a blood vessel or a urinary tract of the patient, etc.) to a remote computer system or processing device.

In some non-limiting embodiments or aspects, smart device 104 may include sensor 254 (e.g., as shown in FIG. 2C) located outside a body of the patient (e.g., at needleless connector 214 at the hub of catheter 702 located outside of a body of the patient, and sensor 254 may be connected to the hub of catheter 702 outside the body of the patient, etc.). For example, sensor 254 may include at least one of a pressure sensor and an acoustic sensor (e.g., a piezoelectric transducer, etc.). As an example, sensor 254 including the pressure sensor and/or the acoustic sensor may be connected to a catheter at needleless connector 214 at the hub of catheter 792. For example, the hub of catheter 702 may include needleless connector 702 and/or smart device 104, and sensor 254 may be included in needleless connector 214. In such an example, sensor 254 may be configured to sense, detect, and/or measure a pressure signal, an acoustic signal, and/or temporal variations in the pressure signal and/or the acoustic signal with the catheter needle in the body of the patient. For example, the pressure signal and/or the acoustic signal sensed by sensor 254 may be transmitted through a fluid in the catheter and/or through material of the catheter (e.g., via needle tip 706, catheter lumen 704, the needleless connector 214, etc.) for sensing by sensor 254. As an example, the pressure signal and/or the acoustic signal sensed by sensor 254 may decrease or drop if needle tip 706 punctures a wall of a blood vessel or urinary tract of the patient. In such an example, a decrease and/or lack in the pressure signal (e.g., a decreased amplitude of a heart rate and/or a drop in blood pressure, etc.) may indicate a lack of a pressure signal associated with an absence of a blood pressure signal, thereby indicating an infiltration event.

In some non-limiting embodiments or aspects, smart device 104 may be programmed and/or configured to compare a relatively slower change or variation in a pressure signal over time (e.g., a relatively slower decrease in an amplitude of a heart rate and/or a drop in blood pressure, etc.) to a threshold level to determine an occlusion event rather than an infiltration event or an extravasation event. For example, an occlusion in a lumen may be at a relatively slow rate over time (e.g., as compared to an infiltration event, an extravasation even, a disconnection event, etc.), which slowly changes the in the pressure signal sensed may sensor 254. As an example, smart device 104 may determine an occlusion even and provide an alter and/or automatically flush a lumen associated with the occlusion in response to detection of the occlusion event. In some non-limiting embodiments, smart device 104 may detect a disconnection event in response to detecting a pressure signal substantially equal to an atmospheric pressure by sensor 254, which indicates that a connection of catheter 702, e.g., needleless connector 214 is disconnected therefrom and provide an alter to a user to address the connection.

In some non-limiting embodiments or aspects, smart device 104 can provide, according the pressure signal and/or the acoustic signal, a location of the tip of the needle with respect to a blood vessel or a urinary tract of the patient in real-time, thereby providing real-time feedback to a user as a catheter is being installed in a blood vessel or a urinary tract of patient to indicate whether the catheter is properly placed within the blood vessel or the urinary tract or if with one of a potential or existing infiltration of the fluid and a potential or existing extravasation of the fluid.

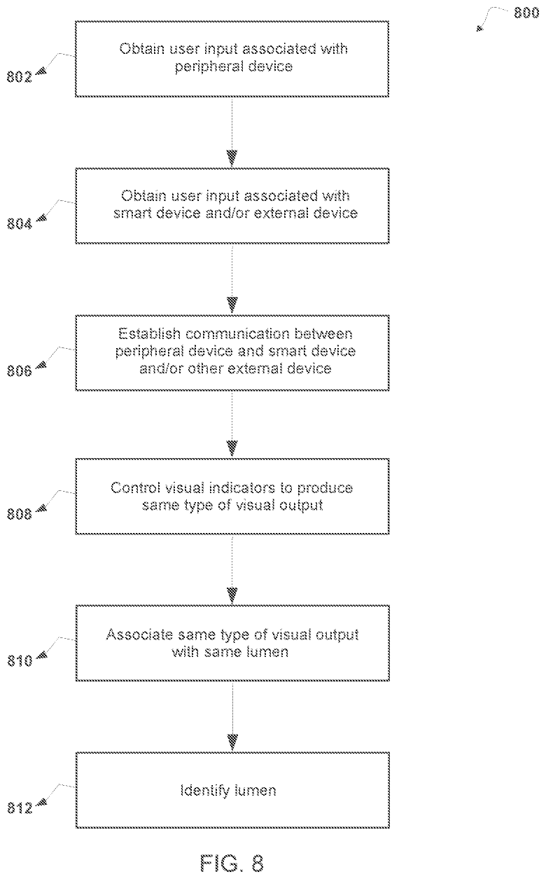

Referring now to FIG. 8, FIG. 8 is a flowchart of a non-limiting embodiment or aspect of a process 800 for identifying a lumen. In some non-limiting embodiments or aspects, one or more of the steps of process 800 are performed (e.g., completely, partially, etc.) by peripheral device 103 (e.g., one or more devices of a system of peripheral device 103, etc.). In some non-limiting embodiments or aspects, one or more of the steps of process 800 are performed (e.g., completely, partially, etc.) by another device or a group of devices separate from or including peripheral device 103, such as medication source system 102 (e.g., one or more devices of medication source system 102, etc.), smart device 104 (e.g., one or more devices of a system of smart device 104, etc.), central computing system 108 (e.g., one or more devices of central computing system 108, etc.), and/or terminal/mobile computing system 110 (e.g., one or more devices of terminal/mobile computing system 110, etc.).

As shown in FIG. 8, at step 802, process 800 includes obtaining user input associated with a peripheral device. For example, peripheral device 103 may obtain user input associated with peripheral device 103. As an example, peripheral device 103 may obtain (e.g., receive, retrieve, determine, etc.) user input received via a user input component (e.g., via pairing input 208, etc.) of peripheral device 103. In such an example, peripheral device 103 may receive data associated with the user input from peripheral device 103.

Referring also to FIG. 2A, in some non-limiting embodiments or aspects, a plurality of peripheral devices 103a, 103b, . . . 103n are connected to a plurality of medication source systems 102a, 102b, . . . 103n that are are connected to a plurality of lumens 212a, 212b, . . . 212n, and each peripheral device 103 may include a visual indicator 210, communication circuitry (e.g., communication interface 314, etc.), and a paring input 208. In some non-limiting embodiments or aspects, each peripheral device 103 receives, via pairing input 208, user input. For example, visual indicator 210 may emit a predetermined light pattern (e.g., blink rapidly and/or emit a predetermined color to indicate that each peripheral device 103 is in a pairing mode, etc.) in response to a predetermined user input to pairing input 208 (e.g., in response to a user pressing and holding a button of pairing input 208, etc.) of each peripheral device 103.

As shown in FIG. 8, at step 804, process 800 includes obtaining user input associated with a smart device (and/or an external device, such as central computing system 108, terminal/mobile computing system 110, and/or the like). For example, peripheral device 103 may obtain user input associated with smart device 104 (and/or an external device, such as central computing system 108, terminal/mobile computing system 110, and/or the like). As an example, peripheral device 103 may obtain (e.g., receive, retrieve, determine, etc.) user input received via a user input component (e.g., pairing input 256, etc.) of smart device 104 (and/or an external device, such as central computing system 108, terminal/mobile computing system 110, and/or the like). In such an example, peripheral device 103 may receive data associated with the user input from smart device 104 that is received at a same time that peripheral device 103 is in the pairing mode.

Referring also to FIGS. 2A and 2B, in some non-limiting embodiments or aspects, a plurality of smart devices 104a, 104b, . . . 104n may be connected (e.g., removably connected, etc.) or configured to be connected to the plurality of lumens 212a, 212b, . . . 212n, and each smart device 104 may include a visual indicator 252, communication circuitry (e.g., communication interface 314, etc.), and a paring input 256. In some non-limiting embodiments or aspects, smart device 104 receives, via pairing input 256 of smart device 104, user input. For example, smart device 104 may establish communication with each peripheral device 103 (e.g., pair and/or activate/initiate a pairing sequence for pairing smart device 104 with each peripheral device 103, etc.) in response to a predetermined user input to paring input 256 (e.g., in response to a user pressing and holding a button of pairing input 256, etc.) of smart device 104 at a same time that each peripheral device 103 is in the pairing mode.

As shown in FIG. 8, at step 806, process 800 includes establishing communication between a peripheral device and a smart device (and/or an external device, such as central computing system 108, terminal/mobile computing system 110, and/or the like). For example, peripheral device 103 may establish communication between peripheral device 103 and smart device 104 (and/or an external device, such as central computing system 108, terminal/mobile computing system 110, and/or the like). As an example, peripheral device 103 may establish communication (e.g., an NFC communication connection, an RFID communication connection, a Bluetooth® communication connection, and/or the like). between peripheral device 103 and smart device 104 (and/or an external device, such as central computing system 108, terminal/mobile computing system 110, and/or the like). In such an example, the communication circuitry of smart device 104 (and/or an external device, such as central computing system 108, terminal/mobile computing system 110, and/or the like) and the communication circuitry of peripheral device 103 may establish the communication between (e.g., pair, etc.) smart device 104 (and/or an external device, such as central computing system 108, terminal/mobile computing system 110, and/or the like) and peripheral device 103 based on the user input received by pairing input 208 of the peripheral device 103 and the user input received by pairing input 256 of smart device 104 (and/or an external device, such as central computing system 108, terminal/mobile computing system 110, and/or the like). For example, peripheral device 103 may establish a short range wireless communication connection (e.g., an NFC communication connection, an RFID communication connection, a Bluetooth® communication connection, etc.) with smart device 104. As an example, visual indicator 210 may be configured to emit a predetermined light pattern (e.g., to blink rapidly to indicate that peripheral device 103 is in a pairing mode, etc.) in response to a predetermined user input to pairing input 208 (e.g., in response to a user pressing and holding a button of pairing input 208, etc.) of peripheral device 103. In such an example, smart device 104 (and/or an external device, such as central computing system 108, terminal/mobile computing system 110, and/or the like) may be configured to establish communication with peripheral device 103 (e.g., pair and/or activate a pairing sequence for pairing smart device 104 (and/or an external device, such as central computing system 108, terminal/mobile computing system 110, and/or the like) with peripheral device 103, etc.) in response to a predetermined user input to paring input 256 (e.g., in response to a user pressing and holding a button of pairing input 256, etc.) of smart device 104 (and/or an external device, such as central computing system 108, terminal/mobile computing system 110, and/or the like) at a same time that peripheral device 103 is in the pairing mode.

As shown in FIG. 8, at step 808, process 800 includes controlling visual indicators of a peripheral device and a smart device to produce a same type of visual output. For example, peripheral device 103, smart device 104, and/or an external device, such as central computing system 108, terminal/mobile computing system 110, and/or the like may control visual indicator 210 of peripheral device 103 and/or visual indicator 252 of smart device 104 (and/or an external device, such as central computing system 108, terminal/mobile computing system 110, and/or the like) to produce a same type of visual output. As an example, peripheral device 103, smart device 104, and/or an external device, such as central computing system 108, terminal/mobile computing system 110, and/or the like may control visual indicator 210 (e.g., a multi-color LED, etc.) of peripheral device 103 and visual indicator 252 (e.g., a multi-color LED, etc.) of smart device 104 (and/or an external device, such as central computing system 108, terminal/mobile computing system 110, and/or the like) to produce a same type of visual output (e.g., a same color of light, the same information associated with a measured parameter of an infusion pump, etc.) based on the communication established between the peripheral device and the smart device (and/or an external device, such as central computing system, terminal/mobile computing system, and/or the like).

In some non-limiting embodiments or aspects, when smart device 104 is paired with peripheral device 103, peripheral device 103 may illuminate visual indicator 210 to a color that has not been previously used (e.g., that is not associated with another peripheral device 103 and another smart device 104 that are paired, that is different than each other color of light produced by each other smart device 104 of the plurality of smart devices 104a, 104b, . . . 104n and each other peripheral device 103 of the plurality of peripheral devices 103a, 103b, . . . 103n, etc.), and smart device 104 may illuminate visual indicator 252 to the same color as visual indicator 210 (e.g., visual indicator 252 may be controlled to illuminate to the same color as visual indictor 210). In some non-limiting embodiments or aspects, smart device 104 may illuminate visual indicator 252 to the same color as visual indicator 210 in response to smart device 104 being connected to a lumen and/or during a period of time at which smart device 104 is connected to the lumen. For example, smart device 104 may automatically stop illumination of visual indicator 252 to the same color as visual indicator 210 (e.g., turn off an LED, set the LED to a default color indicating a non-paired smart device 104, etc.) in response to smart device 104 being disconnected from the lumen. As an example, smart device 104 may include a switch connected to visual indicator 252 and configured to be activated/deactivated in response to a clamp or other connection means being connected/disconnected to a lumen and/or a needleless connector 214 thereof.

In some non-limiting embodiments or aspects, peripheral device 103 (and/or an external device, such as central computing system 108, terminal/mobile computing system 110, and/or the like) determines a color of the same color of light for visual indicator 252 of smart device 104 and visual indicator 210 of peripheral device 103 to produce based on at least one of the user input received by pairing input 208 of peripheral device 103 and the user input received by pairing input 256 of smart device 104. For example, after smart device 104 (and/or an external device, such as central computing system 108, terminal/mobile computing system 110, and/or the like) is paired with peripheral device 103, a user may actuate pairing input 208 and/or pairing input 256 to cycle through colors of light available for the pairing to select a desired (and/or available or previously unused) color of light for the pairing.

As shown in FIG. 8, at step 810, process 800 includes associating a same type of visual output with a same lumen. For example, peripheral device 103 (and/or an external device, such as central computing system 108, terminal/mobile computing system 110, and/or the like) may associate (e.g., automatically associate, etc.) a same type of visual output with a same lumen. As an example, peripheral device 103 (and/or an external device, such as central computing system 108, terminal/mobile computing system 110, and/or the like) may associate (e.g., store in connection with, pair, link, illuminate with, etc.) the same type of visual output (e.g., a same color of light, the same information associated with a measured parameter of an infusion pump, etc.) with a same lumen (e.g., with a same lumen of a plurality of lumens 212a, 212b, . . . 212n, etc.) and/or a same medication source device (e.g., with a same medication source device of a plurality of medication source devices 206a, 206b, . . . 206n, etc.). In such an example, peripheral device 103 and smart device 104 (and/or an external device, such as central computing system 108, terminal/mobile computing system 110, and/or the like) may be connected to the same lumen. Accordingly, a user may more easily identify a lumen or line, a location of the lumen or line, a medication that has been or is being delivered via the lumen or line, which infusion pump or mediation source to which the lumen or line is connected, which lumens or lines are available for medication delivery based on compatibility with previous and/or ongoing medication deliveries, and/or the like.

In some non-limiting embodiments or aspects, peripheral device 103 may obtain user input received by a user input component of another peripheral device, obtain user input received by a user input component of another smart device (and/or an external device, such as central computing system 108, terminal/mobile computing system 110, and/or the like), establish a communication between the another peripheral device and the another smart device (and/or an external device, such as central computing system 108, terminal/mobile computing system 110, and/or the like) based on the user input received by the user input component of the another peripheral device and the user input received by the user input component of the another smart device, control the visual indicator of the another smart device (and/or an external device, such as central computing system 108, terminal/mobile computing system 110, and/or the like) and the visual indicator of the another peripheral device to produce another same type of visual output based on the communication established between the another peripheral device and the another smart device, wherein the another same type of visual output is different than the same type of visual output, and/or associate the another same type of visual output with another same lumen of the plurality of lumens, wherein the another peripheral device is connected to another infusion pump connected to the another same lumen. For example, and referring again to FIG. 2A, peripheral device 103a may be paired with smart device 104n and each of peripheral device 103a and smart device 104n may output a first color of light (e.g., red light) associated with lumen 212a, peripheral device 103b may be paired with smart device 104a and each of peripheral device 103b and smart device 104a may output a second color of light (e.g., green light) associated with lumen 21b, peripheral device 103n may be paired with smart device 104b and each of peripheral device 103n and smart device 104b may output a third color of light (e.g., blue light) associated with lumen 212n, and/or the like.

As shown in FIG. 8, at step 812, process 800 includes identifying a lumen. For example, peripheral device 103 (and/or an external device, such as central computing system 108, terminal/mobile computing system 110, and/or the like) may identify a lumen. As an example, peripheral device 103 (and/or an external device, such as central computing system 108, terminal/mobile computing system 110, and/or the like) may identify the same lumen associated with the same type of visual output.

In some non-limiting embodiments or aspects, peripheral device 103 (and/or an external device, such as central computing system 108, terminal/mobile computing system 110, and/or the like) identifies a lumen by automatically associating and/or providing medical data with the same type of visual output associated with the lumen and/or an identifier of the lumen. For example, medical data may include at least one of the following: information associated with a measured parameter of an infusion pump, a state of an infusion pump, an identifier of a peripheral device 103, patient data (e.g., an identifier of a particular patient, information and/or data associated with a patient, etc.); medication source data (e.g., an identifier of a particular medication source device 206, etc.); medication data (e.g., an identifier of a type of a medication, a scheduled delivery of a particular medication, a previous delivery of a particular medication, a lumen associated with a medication, etc.); lumen data (e.g., an identifier of a particular lumen, such as the identifier of the same lumen associated with the same type of visual output, etc.); sensor data (e.g., an identifier of a particular sensor 254, information, data, and/or a signal sensed, measured, and/or detected by one or more sensors 254 in one or more smart devices 104, etc.); compliance data (e.g., information or data associated with a scrubbing event in which a needleless connector 214 and/or a lumen is scrubbed with a disinfectant, information or data associated with a flushing event in which a needleless connector 214 and/or a lumen is flushed with a solution, information or data associated with a connection or capping event in which a needleless connector 214 or a lumen is connected to a medical device, etc.); location data (e.g., a location of a patient, a location a previous or scheduled fluid delivery procedure, a location a lumen, a location of a medication source device, etc.); time data (e.g., a time associated with a previous or scheduled fluid delivery procedure, a time of connection of a lumen to medication source device 206, a time of connection of smart device 104 to a lumen, a time of pairing of medication source device 206 and smart device 104, etc.); a location of a tip of a needle of a catheter of a lumen with respect to a blood vessel or urinary tract of the patient; or any combination thereof. As an example, peripheral device 103 (and/or an external device, such as central computing system 108, terminal/mobile computing system 110, and/or the like) may obtain medical data from smart device 104, central computing system 108, terminal/mobile computing system 110, one or more databases connected thereto, and/or one or more sensors (e.g., a barcode sensor for scanning a patient identifier, a fluid flow sensor for sensing a flow a fluid, a pressure sensor for sensing a pressure in an IV line, a medication type sensor for sensing a type of a medication, etc.) connected thereto. In such an example, peripheral device 103 (and/or an external device, such as central computing system 108, terminal/mobile computing system 110, and/or the like) may identify lumens with information and/or data associated therewith, as well as provide a visual indication of which lumens of a plurality of lumens 212a, 212b, . . . 212n are connected to which medication source devices of a plurality of medication source devices 206a, 206b, . . . 206n, which can enable a user to more easily trace a lumen from a patient to a particular medication source device to which the lumen is connected; connections between lumens and medication source devices to be removed if the patient is moved (e.g., to a new room, to a new floor, to surgery, to the bathroom, etc.) with the same type of visual indicator on a lumen/medication source device pair used to more easily reattach the correct medication source device channel to the correct (e.g., the same as before) lumen; tracking compliance to best practice protocols, for example, by determining if hub scrubbing has occurred and if hub scrubbing occurred effectively (e.g., sufficient pressure, sufficient time scrubbing, etc.) and/or if a device has been flushed, maintained, and/or the like; providing reminders and prescriptive help for protocol adherence, and/or the like.

In some non-limiting embodiments or aspects, peripheral device 103 (and/or an external device, such as central computing system 108, terminal/mobile computing system 110, and/or the like) identifies a lumen by determining and providing, based on the medical data, one or more alerts or reminders associated with the lumen and/or the same type of visual output associated with the lumen, such as a reminder to flush the lumen and/or a needleless connector 214 thereof, a reminder to remove or replace a lumen, med-mined infection prevention guidance, an alert to use a different lumen for delivery of a particular medication to reduce a chance of a chemical occlusion forming, an alert indicating whether to treat a lumen for thrombus occlusion or chemical occlusion, an alert that an occlusion is detected in a lumen, an alert that a location of a tip of a needle connected to the lumen is associated with one of a potential or existing infiltration of the fluid and a potential or existing extravasation of the fluid, and/or the like.

In some non-limiting embodiments or aspects, peripheral device 103 (and/or an external device, such as central computing system 108, terminal/mobile computing system 110, and/or the like) identifies a lumen by controlling a medication source device 206 or another medical device (e.g., an electronic valve, etc.), based on the medical data, to inhibit or prevent delivery of a fluid (e.g., a particular medication, a type of medication, etc.) via the lumen.

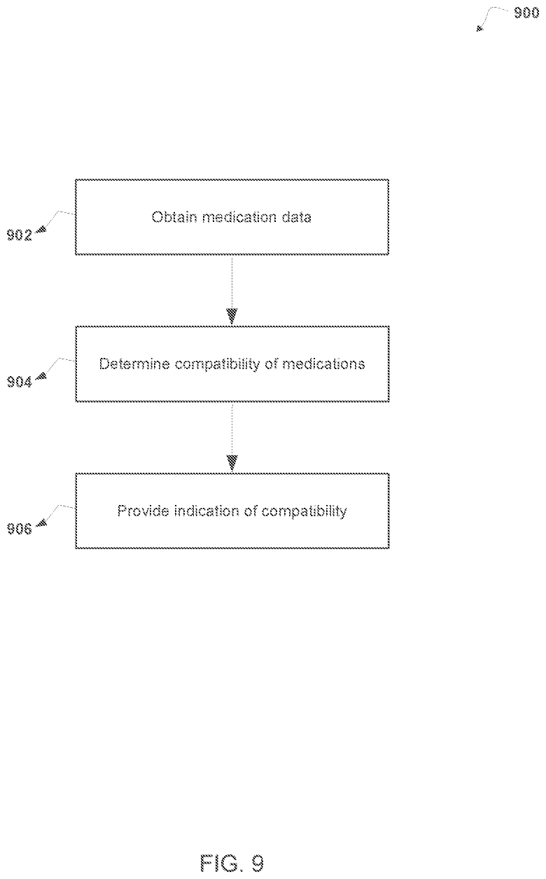

Further details regarding non-limiting embodiments or aspects of step 812 of process 800 are provided below with regard to FIG. 9.

Referring now to FIG. 9, FIG. 9 is a flowchart of a non-limiting embodiment or aspect of a process 900 for identifying a lumen. In some non-limiting embodiments or aspects, one or more of the steps of process 900 are performed (e.g., completely, partially, etc.) by peripheral device 103 (e.g., one or more devices of a system of peripheral device 103, etc.). In some non-limiting embodiments or aspects, one or more of the steps of process 900 are performed (e.g., completely, partially, etc.) by another device or a group of devices separate from or including peripheral device 103, such as smart device 104 (e.g., one or more devices of a system of smart device 104, etc.), central computing system 108 (e.g., one or more devices of central computing system 108, etc.), and/or terminal/mobile computing system 110 (e.g., one or more devices of terminal/mobile computing system 110, etc.).

As shown in FIG. 9, at step 902, process 900 includes obtaining medication data. For example, peripheral device 103 (and/or an external device, such as central computing system 108, terminal/mobile computing system 110, and/or the like) may obtain medication data. As an example, peripheral device 103 (and/or an external device, such as central computing system 108, terminal/mobile computing system 110, and/or the like) may obtain medication data associated with a first type of medication delivered or scheduled to be delivered via the same lumen to a patient and a second type of medication delivered or scheduled to be delivered via the same lumen to the patient. In such an example, the first type of medication may be different than the second type of medication.

In some non-limiting embodiments or aspects, medication data is associated with at least one of the following: an identifier of a type of a medication, a scheduled delivery of the medication via a particular medication source device, and/or lumen, a previous delivery of the medication via a particular medication source device and/or lumen, an amount of the medication, an identifier of a patient to which the medication is scheduled to be delivered (or delivered), one or more identifiers of one or more different types of medication that are incompatible for delivery via a same lumen with the medication, and/or the like.

As shown in FIG. 9, at step 904, process 900 includes determining compatibility of medications. For example, peripheral device 103 (and/or an external device, such as central computing system 108, terminal/mobile computing system 110, and/or the like) may determine compatibility of medications. As an example, medication source system 102 may determine, based on the medication data, a compatibility of the second type of medication for delivery via the same lumen as the first type of medication.

In some non-limiting embodiments or aspects, peripheral device 103 (and/or an external device, such as central computing system 108, terminal/mobile computing system 110, and/or the like) may use an identifier of the first type of medication and/or an identifier of the second type of medication to access a look-up table that indicates whether the first type of medication and the second type of medication are compatible or incompatible (e.g., compatible or incompatible for delivery via a same lumen, etc.). In some non-limiting embodiments or aspects, the look-up table maybe be stored in and/or associated with the identifier of the first type of medication and/or the identifier of the second type of medication.

In some non-limiting embodiments or aspects, peripheral device 103 (and/or an external device, such as central computing system 108, terminal/mobile computing system 110, and/or the like) may obtain medication data associated with a third type of medication delivered or scheduled to be delivered via another same lumen (e.g., different than the same lumen, etc.) to the patient, and determine, based on the medication data, a compatibility of the second type of medication for delivery via the another same lumen as the third type of medication, wherein the indication further indicates whether the second type of medication is compatible for delivery via the another same lumen associated with the another same type of visual output. For example, and referring again to FIGS. 2A and 2B, if peripheral device 103 (and/or an external device, such as central computing system 108, terminal/mobile computing system 110, and/or the like) determines that the second type of medication is incompatible for delivery via a first lumen 212a, peripheral device 103 (and/or an external device, such as central computing system 108, terminal/mobile computing system 110, and/or the like) may determine a compatibility of the second type of medication for delivery via an alternative lumen, such as a second lumen 212b based a third type of medication delivered or scheduled to be delivered via the second lumen 212b and, if the second type of medication is compatible for delivery via the same lumen as the third type of medication, provide the indication that the second type of medication is compatible for delivery via the second lumen 212b.

As shown in FIG. 9, at step 906, process 900 includes providing an indication of compatibility. For example, peripheral device 103 (and/or an external device, such as central computing system 108, terminal/mobile computing system 110, and/or the like) may provide an indication of compatibility. As an example, peripheral device 103 (and/or an external device, such as central computing system 108, terminal/mobile computing system 110, and/or the like) may provide an indication of whether the second type of medication is compatible for delivery via the same lumen associated with the same type of visual output. As another example, peripheral device 103 (and/or an external device, such as central computing system 108, terminal/mobile computing system 110, and/or the like) may provide an indication of whether the third type of medication is compatible for delivery via the another same lumen associated with the another same time of visual output.

In some non-limiting embodiment or aspects, peripheral device 103 (and/or an external device, such as central computing system 108, terminal/mobile computing system 110, and/or the like) may provide the indication of the compatibility by controlling medication source device 206 to inhibit or prevent delivery of the second medication via the same lumen associated with the same type of visual output. For example, the first type of medication may be delivered to the patient with the same lumen associated with the same type of visual output, and the second type of medication may be scheduled to be delivered via the same lumen to the patient. As an example, and referring again to FIGS. 2A and 2B, peripheral device 103 (and/or an external device, such as central computing system 108, terminal/mobile computing system 110, and/or the like) may determine, based on the medical data including the medication data, that a first type of drug is delivered via lumen 212a to the patient and that a second type of drug that is scheduled for delivery or attempting to be delivered via the same lumen 212a is incompatible with the first type of drug (e.g., likely to cause an occlusion, likely to cause an adverse reaction in the patient, etc.). In such an example, peripheral device 103 (and/or an external device, such as central computing system 108, terminal/mobile computing system 110, and/or the like) may control medication source device 206a to inhibit or prevent delivery of the second medication via the same lumen 212a (e.g., by stopping a pump, closing a valve, etc.) and/or providing a prompt to the user to use another lumen (e.g., 212b, . . . 212n, etc.) associated with a different type of visual output than the same type of visual output to deliver the second type of medication to the patient.

In some non-limiting embodiments or aspects, the first type of medication and the second type of medication may be delivered to the patient via the same lumen associated with the same type of visual output, and peripheral device 103 (and/or an external device, such as central computing system 108, terminal/mobile computing system 110, and/or the like) may provide a prompt to the user to treat the same lumen associated with the same type of visual output for one of a thrombus occlusion and a chemical occlusion. For example, when an occlusion occurs, which may be detected as described herein, a user (e.g., a nurse, etc.) may need to determine if the occlusion is thrombotic or chemical due to drug interactions, and peripheral device 103 (and/or an external device, such as central computing system 108, terminal/mobile computing system 110, and/or the like) can determine which medications were delivered via which lumens to inform the user of the lumen history and/or provide an indication of a potential cause of the occlusion, which enables a correct decision of whether the lumen should be treated for thrombus or chemical occlusion. In some non-limiting embodiments or aspects, peripheral device 103 (and/or an external device, such as central computing system 108, terminal/mobile computing system 110, and/or the like) may control medication source device 206 to automatically perform a flushing operation to deliver a flushing fluid to a lumen connected to the medication source device 206 in response to a determination that an occlusion of the lumen is a chemical occlusion.

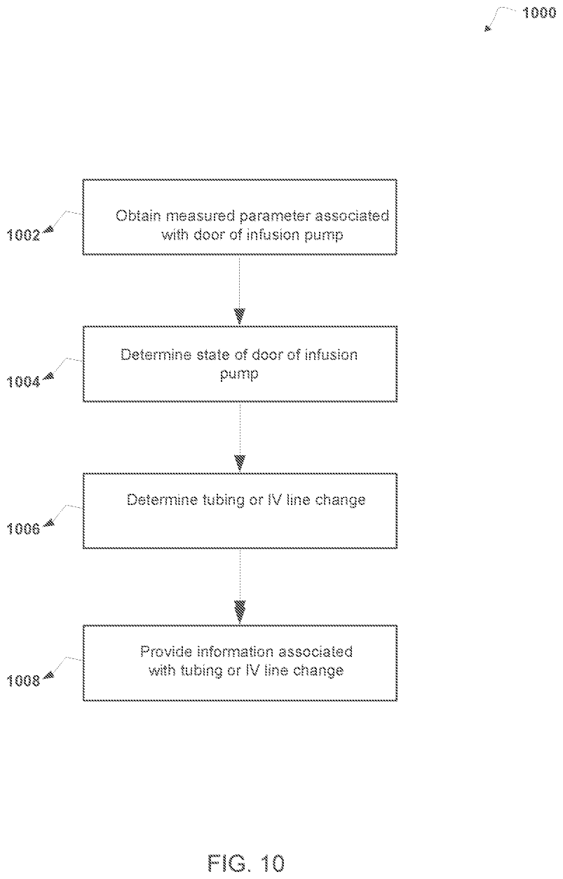

Referring now to FIG. 10, FIG. 10 is a flowchart of a non-limiting embodiment or aspect of a process 1000 for determining a tubing or IV line change. In some non-limiting embodiments or aspects, one or more of the steps of process 1000 are performed (e.g., completely, partially, etc.) by peripheral device 103 (e.g., one or more devices of a system of peripheral device, etc.). In some non-limiting embodiments or aspects, one or more of the steps of process 1000 are performed (e.g., completely, partially, etc.) by another device or a group of devices separate from or including peripheral device 103, such as medication source system 102 (e.g., one or more devices of medication source system 102, etc.), smart device 104 (e.g., one or more devices of a system of smart device 104, etc.), central computing system 108 (e.g., one or more devices of central computing system 108, etc.), and/or terminal/mobile computing system 110 (e.g., one or more devices of terminal/mobile computing system 110, etc.).

As shown in FIG. 10, at step 1002, process 1000 includes obtaining a measured parameter associated with a door of an infusion pump. For example, peripheral device 103 may obtain a measured parameter associated with a door of infusion pump 206. As an example, peripheral device 103 may include at least one of the following: a button configured for mechanical interaction with a door of the infusion pump (e.g., a door used to access a connection between an IV line and the infusion pump, etc.), a piezoelectric sensor, a tilt switch, an accelerometer, or any combination thereof, configured to obtain a corresponding measured parameter associated with the door of the infusion pump 206.

As shown in FIG. 10, at step 1004, process 1000 includes determining, based on a measured parameter associated with a door of an infusion pump, a state of the door of the infusion pump. For example, peripheral device 103 may determine, based on the measured parameter associated with the door of infusion pump 206, a state of the door of the infusion pump (e.g., one or more states or state changes over a period of time, a series or pattern of state changes over a period of time, a door open state, a door closed state, etc.).

As shown in FIG. 10, at step 1006, process 1000 includes determining, based on a state of a door of the infusion pump, that a tubing or IV line change for the infusion pump has been performed. For example, peripheral device 103 may determine, based on the door open state and/or the door closed state of the door of infusion pump 206 (e.g., over time, etc.), that a tubing or IV line change for infusion pump 206 has been performed. In such an example, an opening and closing of the door may indicate that a tubing or IV line change for infusion pump 206 has been performed.

As shown in FIG. 10, at step 1008, process 1000 includes providing information associated with a tubing or IV line change for an infusion pump. For example, peripheral device 103 (and/or an external device, such as central computing system 108, terminal/mobile computing system 110, and/or the like) may provide information associated with the determined tubing or IV line change for an infusion pump and/or a reminder to a clinician regarding when a tubing or IV line change is scheduled to be performed.

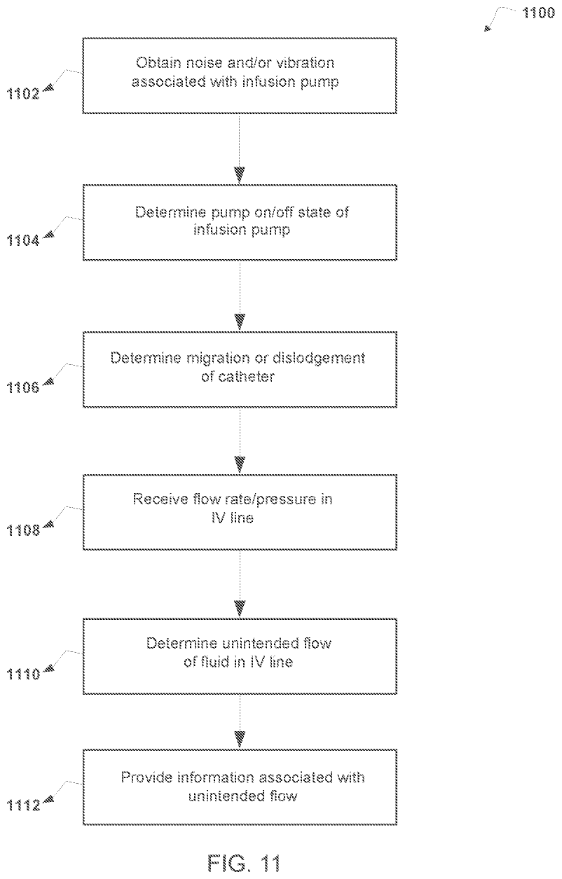

Referring now to FIG. 11, FIG. 11 is a flowchart of a non-limiting embodiment or aspect of a process 1100 for event monitoring. In some non-limiting embodiments or aspects, one or more of the steps of process 1100 are performed (e.g., completely, partially, etc.) by peripheral device 103 (e.g., one or more devices of a system of peripheral device 103, etc.). In some non-limiting embodiments or aspects, one or more of the steps of process 1100 are performed (e.g., completely, partially, etc.) by another device or a group of devices separate from or including peripheral device 103, such as medication source system 102 (e.g., one or more devices of medication source system 102, etc.), by smart device 104 (e.g., one or more devices of a system of smart device 104, etc.), central computing system 108 (e.g., one or more devices of central computing system 108, etc.), and/or terminal/mobile computing system 110 (e.g., one or more devices of terminal/mobile computing system 110, etc.).

As shown in FIG. 11, at step 1102, process 1100 includes obtaining a noise and/or vibration associated with an infusion pump. For example, peripheral device 103 may obtain a noise and/or vibration associated with infusion pump 206. As an example, peripheral device 103 may obtain a noise and/or vibration signal or signature measured by sensor 280 (e.g., an acoustic and/or vibration sensor, etc.). As an example, sensor 280 of peripheral device 103 may include an acoustic or vibration sensor configured to measure a noise or vibration of the infusion pump.

As shown in FIG. 11, at step 1104, process 1100 includes determining, based on a measured noise or vibration of an infusion pump, a pump on state and a pump off state of the infusion pump. For example, peripheral device 103 may determine, based on the measured noise or vibration of infusion pump 206, a pump on state and/or a pump off state of infusion pump. As an example, peripheral device 103 may compare the measured noise or vibration of infusion pump 206 to one or more reference noise or vibration signatures associated with a pump on state, a pump off state, an unintended flow, a catheter migration or dislodgement, or any combination thereof.

As shown in FIG. 11, at step 1106, process 1100 includes determining, based on a measured noise or vibration of an infusion pump, a migration or a dislodgement of an IV line connected to the infusion pump. For example, peripheral device 103 may determine, based on the measured noise or vibration of infusion pump 206, a migration or a dislodgement of an IV line connected to infusion pump 206. As an example, peripheral device 103 may compare the measured noise or vibration of infusion pump 206 to one or more reference noise or vibration signatures associated with a pump on state, a pump off state, an unintended flow, a catheter migration or dislodgement, or any combination thereof, to determine the state of infusion pump 206 and/or one or more components connected thereto. In such an example, peripheral device 103 may determine, based on the measured noise or vibration of infusion pump 206, a migration or a dislodgement of an IV line connected to infusion pump 206.

As shown in FIG. 11, at step 1108, process 1100 includes receiving at least one of a flow rate and a pressure of a fluid in an IV line connected to an infusion pump. For example, peripheral device 103 may receive, from smart device 104, at least one of a flow rate and a pressure of a fluid in the IV line connected to infusion device 206.

As shown in FIG. 11, at step 1110 process 1100 includes determining, based on a measured noise or vibration of an infusion pump and at least one of a flow rate and a pressure of a fluid in an IV line connected to the infusion pump, an unintended flow of the fluid in the IV line. For example, peripheral device 103 may determine, based on the measured noise or vibration of the infusion pump and the at least one of the flow rate and the pressure of the fluid in the IV line, an unintended flow of the fluid in the IV line. As an example, smart device 104 may be connected to the IV line connected to infusion pump 206, and smart device 104 may measure at least one of a flow rate and a pressure of a fluid in the IV line and provide the measured flow rate and/or pressure to peripheral device 103 (and/or smart device 104, central computing system 108, terminal/mobile computing system 110, etc.). In such an example, peripheral device 103 (and/or smart device 104, central computing system 108, terminal/mobile computing system 110, etc.) may determine, based on the measured noise or vibration of the infusion pump and the at least one of the flow rate and the pressure of the fluid in the IV line, an unintended flow of the fluid in the IV line.

As shown in FIG. 11, at step 1112 process 1100 includes providing information associated with an unintended flow. For example, peripheral device 103 (and/or an external device, such as central computing system 108, terminal/mobile computing system 110, and/or the like) may provide information associated with the determined unintended flow in the IV line connected to the infusion pump.

Further details regarding non-limiting embodiments or aspects of devices, systems, methods, and computer program products described herein are disclosed in Appendix A filed herewith.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A peripheral device for an infusion pump, comprising:
a housing including:
a sensor configured to measure a parameter associated with the infusion pump;
a wireless communication device configured to communicate, to an external device, information associated with the measured parameter of the infusion pump; and
a mechanical interface configured to connect the housing to an exterior surface of the infusion pump.

2. The peripheral device of claim 1, wherein the housing is removably connected to the mechanical interface.

3. The peripheral device of claim 1, wherein the housing is integrated with the mechanical interface.

4. The peripheral device of claim 1, wherein the mechanical interface includes a base plate configured to be connected to the exterior surface of the infusion pump.

5. The peripheral device of claim 1, wherein the mechanical interface includes an adhesive layer.

6. The peripheral device of claim 1, wherein the mechanical interface includes a snap fit connector.

7. The peripheral device of claim 1, wherein the housing further includes a battery configured to provide power to the sensor and the wireless communication device.

8. The peripheral device of claim 7, wherein the battery includes a rechargeable battery.

9. The peripheral device of claim 1, wherein the housing includes a cover and a base, and wherein the cover is rotatable about an pin connecting the cover to the base.

10. The peripheral device of claim 1, wherein the sensor includes at least one of the following: a button configured for mechanical interaction with a door of the infusion pump, a piezoelectric sensor, a tilt switch, an accelerometer, or any combination thereof.

11. The peripheral device of claim 10, wherein the housing further includes one or more processors programmed and/or configured to determine, based on the measured parameter, a door open state and a door closed state of the door of the infusion pump.

12. The peripheral device of claim 1, wherein the sensor includes an acoustic or vibration sensor configured to measure a noise or vibration of the infusion pump.

13. The peripheral device of claim 12, wherein the housing further includes one or more processors programmed and/or configured to determine, based on the measured noise or vibration of the infusion pump, a pump on state and a pump off state of the infusion pump.

14. The peripheral device of claim 13, wherein the wireless communication device is configured to receive, from a smart device connected to an IV line connected to the infusion pump, sensor data associated with at least one of a flow rate and a pressure of a fluid in the IV line, and wherein the one or more processors are programmed and/or configured to determine, based on the measured noise or vibration of the infusion pump and the at least one of the flow rate and the pressure of the fluid in the IV line, an unintended flow of the fluid in the IV line.

15. The peripheral device of claim 13, wherein the housing further includes one or more processors programmed and/or configured to determine, based on the measured noise or vibration of the infusion pump, a migration or a dislodgement of an IV line connected to the infusion pump.

16. The peripheral device of claim 1, wherein the housing further includes:
a user input component configured to receive a user input from a user.

17. The peripheral device of claim 1, wherein the housing further includes a user feedback component configured to provide feedback to the user.

18. The peripheral device of claim 17, wherein the user feedback component is configured to produce a same type of visual output as a smart device connected to an IV line connected to the infusion pump based on a communication connection established between the wireless communication device and the smart device.

19. The peripheral device of claim 1, wherein the housing further includes an identifier element encapsulating a unique identifier associated with the peripheral device, and wherein the identifier element includes at least one of the following: a barcode, an RFID tag, or any combination thereof.

20. A system comprising:
the peripheral device of claim 1; and
the infusion pump, wherein the peripheral device is connected to the infusion pump.

21.-45. (canceled)
